(19) United States
(12) Patent Application Publication
(10) Pub. No.: US 2017/0137833 A1
(43) Pub. Date: May 18, 2017

(54) AGROBACTERIUM AND METHOD FOR PRODUCING TRANSFORMED PLANT USING THE SAME

(71) Applicant: KANEKA CORPORATION, Osaka-shi (JP)

(72) Inventors: Haruyasu HAMADA, Takasago-shi (JP); Yozo NAGIRA, Takasago-shi (JP); Naoaki TAOKA, Takasago-shi (JP); Ryozo IMAI, Sapporo-shi (JP); Mineo KOJIMA, Nagoya-shi (JP); Ryuji MIKI, Takasago-shi (JP)

(73) Assignee: KANEKA CORPORATION, Osaka-shi (JP)

(21) Appl. No.: 15/311,435

(22) PCT Filed: May 15, 2015
(86) PCT No.: PCT/JP2015/063990

§ 371 Date: Nov. 15, 2016

(30) Foreign Application Priority Data
May 16, 2014 (JP) 2014-102566

(51) Int. Cl. C12N 15/82

(52) U.S. Cl. CPC C12N 15/8205

(57) ABSTRACT

It is an object solved by the present invention to provide an Agrobacterium with increased gene targeting efficiency. The present invention provides an Agrobacterium having a T-DNA nuclear translocation ability equivalent to that of a wild-type Agrobacterium and also having a T-DNA chromosomal insertion ability that is lost or reduced in comparison to a wild-type Agrobacterium.

### TECHNICAL FIELD

The present invention relates to Agrobacterium with inceased gene targeting efficiency, and a method for producing a transformed plant using the same.

### BACKGROUND ART

In the currently popular plant transformation technique (Non Patent Literature 1), a gene of interest cannot be inserted into a targeted site on the chromosome, but it can be randomly inserted into a site on the chromosome. In addition, the copy number of genes of interest to be introduced cannot be regulated. Thereby, the current transformation technique is problematic, for example, in that a variation in the gene expression level depending on the insertion site of a foreign gene, namely, “position effect” is generated (Non Patent Literature 2), in that a gene of interest is inserted into a position on the chromosome that affects non growth, and in that insertion of a plurality of genes of interest causes inactivation of the genes of interest. Hence, in the current transformation technique, it is necessary to produce an enormous number of transformants, and then to select transformants, in which a gene of interest has been inserted into only a desired site. Thus, the current transformation technique is problematic in terms of poor efficienecy. In order to overcome this problem and to produce safe and highly-functional, genetically-modified crops, it has been desired to develop a gene targeting technique capable of introducing a desired copy number of genes of interest into designated positions on the chromosome.

In recent years, as gene targeting techniques in plants that have been developed, methods of utilizing artificial nuclease, modification of a chromatin structure, or homologous recombination-related factors and the like have been reported (Non Patent Literature 3 to 5). These are all techniques of modifying a plant body to improve homologous recombination efficiency, and are not highly versatile in that optimization is required for each plant species to which the techniques are applied. Moreover, these techniques are also problematic in that the combined use with other techniques is difficult.

On the other hand, a Vir gene has been known as an Agrobacterium gene associated with insertion of a gene of interest into a site on the plant chromosome in the transformation of a plant using Agrobacterium. Patent Literature 1 discloses a method of allowing a protein to transiently express in a plant, and this publication also describes that a wild-type Agrobacterium strain (e.g., Agrobacterium tumefaciens, etc.), or an Agrobacterium strain in which one or several genes are mutated and transformation efficiency is thereby increased (e.g., an Agrobacterium strain comprising redundant virG gene copies, such as a pTiBo542-derived super VirG gene preferably linked to a multiple-copy plasmid, such as, for example, an Agrobacterium strain, in which vir gene expression and/or induction thereof are modified by the presence of a mutant virA gene or virG gene), is used as Agrobacterium (paragraph [0127]). Patent Literature describes the use of an Agrobacterium strain comprising T-DNA containing a gene encoding a functional virE2 protein and a desired gene, together with an Agrobacterium strain comprising T-DNA containing a selective marker gene and having no ability to generate a functional virE2 protein, for simultaneous transformation of plant cells and the above-described two T-DNAs (Claim 10). Moreover, Patent Literature 3 discloses that the T-DNA delivery efficiency of an Acrobacterium deleting virE2 is approximately 10,000 times lower than that of a wild-type Agrobacterium or a mutant Agrobacterium supplemented with trans isomers (paragraph [0069]).

### PRIOR ART LITERATURES

### Patent Literatures

Patent Literature 1: JP Patent Publication (Kohyo) No. 2013-534430 A
Patent Literature 2: JP Patent Publication (Kohyo) No. 2011-505806 A
Patent Literature 3: JP Patent Publication (Kohyo) No. 2008-505622 A

### Non Patent Literatures

Non Patent Literature 1: Pitzschke and Hirt. New insights into an old story: Agrobacterium-induced tunor formation in plants by plant transformation. The EMBO Journal (2010) 29, 1021-1032
Non Patent Literature 2: Matzke A. and Matzke M. (1998) Position effects and epigenetic silencing of plant transgenes, Curr. 0pin. Plant Biol. 1 142-148.
Non Patent Literature 3: Kumar et al, “Gene Targeting: Development of Novel Systems for Genome Engineering in Plants” Floriculture, Ornamental and Plant Biotechnology Volume IV (2006) Global Science Books
Non Patent Literature 4: Shukla, et al. “Precise genome modification in the crop species Zea mays using zinc-finger nucleases.” Nature (2009) 459 437-441
Non Patent Literature 5: Endo, et al. Increased frequency of homologous recombination and T-DNA integration in Arabidopsis CAF-1 mutants, EMBO J. 25 (2006)5579-5590,

### SUMMARY OF INVENTION

### Object to be Solved by the Invention

It Is an object solved by the present invention to provide a plant transformation technique that is highly versatile and can be used in combination with other plant transformation techniques. More specifically, it is an object solved by the present invention to provide an Agrobacterium with increased gene targeting efficiency. It is another object solved by the present invention to provide a method for producing the aforementioned Agobacterium, plant cells obtained using the aforementioned Agrobacterium, and a method for producing a transformed plant using the aforementioned Agrobacterium.

### Means for Solving the Object

As a result of intensive studies directed towards achieving the aforementioned objects, the present inventors have found that gene targeting efficiency can be significantly increased by transforming a plant with an Agrobacterium having a T-DNA nuclear translocation ability equivalent to that of a wild-type Agrobacterium and also having a T-DNA chromosomal insertion ability that is lost or reduced in comparison to a wild-type Agrobacterium, thereby completing the present invention.

Thus, the present invention provides the following invention.
[1] An Agrobacterium, which has a T-DNA nuclear translocation ability equivalent to that of a wild-type Agrobacterium, and has a T-DNA chromosomal insertion ability that is lost or reduced in comparison to a wild-type Agrobacterium.
[2] The Agrobacterium according to [1], wherein homologous recombination efficiency upon its transformation in a plant is two or more times increased in comparison to a wild-type Agrobacterium, wherein the homologous recombination efficency means the number of plants undergoing homologous recombination/the number of plants subjected to transformation.
[3] The Agrobacterium according to [1] or [2], wherein gene targeting efficiency upon its transformation in a plant is two or more times increased in comparison to a wild-type Agrpbaaterium, wherein the gene targeting efficiency means homologous recombination efficiency/random recombination efficiency, the homologous recombination efficiency means the number of plants undergoing homologous recombination/the number of plants subjected to transformation, and the random recombination efficiency means the number of plants undergoing random recombination/the number of plants subjected to transformation.
[4] The Agrobacterium according to any one of [1] to [3], wherein the gene targeting efficiency upon its transformation in a plant is 1% or more, wherein the gene targeting efficiency means homologous recombination efficency/random recombination efficiency, the homologous recombination efficiency means the number of plants undergoing homologous recombination/the number of plants subjected to transformation, and the random recombination efficiency means the number of plants undergoing random recombination/the number of plants subjected to transformation.
[5] The Agrobacterium according to any one of [1] to [4], wherein the function of a gene associated with T-DNA random insertion is lost or reduced.

The Agrobacterium according to [5], wherein the gene associated with T-DNA random insertion is one or more selected from among an ATU3081 gene, an ATU309 gene, an ATU6150 (virH1) gene, an ATU6154 (virF) gene, an ATU1656 (virK) gene, an ATU6183 (virD3) gene, an ATU6184 (virD4) gene, an ATU6185 (virD5) gene, an ATU6188 (virE0) gene, an ATU6189 (virE1) gene, an ATU6191 (virE3) gene, ATU6180 (virC1) gene, and a homologous gene thereof.
[7] The Agrobacterium according to one of [1] to [6], which is Agrobacterium rhizogenes or Agrobacterium tumefaciens.
[8] The Agrobacterium according to any one of [1] to [7], which has a targeting vector comprising a foreign gene.
[9] A method for producing the Agrobacterium according to any one of [1] to [8], which comprises disrupting a gene associated with T-DNA random insertion in the chromosome of an Agrobacterium by a homologous recombination method.
[10] A plant cell which is obtained by infecting a plant cell with the Agrobacterium according to [8].
[11] A method for producing a transformed plant, which comprises infecting a plant cell with the Agrobacterium according to [8].

### Advantageous Effects of Invention

According to the present invention, gene targeting efficiency (homologous recombination efficiency) can be significantly increased by modifying an Agrobacterium gene that mediates transformation. According to the method of the present invention, by utilizing homologous recombination, a gene can be introduced with high efficiency into a specific site on the chromosome of a plant.

### BRIEF DESCRIPTION OF DRAWINGS

FIG. 1 shows confirmation of the gene disruption of an Agrobacterium by genomic PCR. W: a wild-type Agrobacterium strain, and M: an Agrobacterium gene disrupted strain.

FIG. 2 shows a gene targeting evaluation system is Arabidopsis thaliana.

FIG. 3 shows T1 seeds that emit GFP fluorescence.

### EMBODIMENTS FOR CARRYING OUT THE INVENTION

Hereinafter, the present invention will be described in detail.

The present invention relates to an Agrobacterium having T-DNA nuclear translocation ability equivalent to that of a wild-type Agrobacterium and also having a T-DNA chromosomal insertion ability that is lost or reduced in comparison to a wild-type Agrobacterium. By transforming a plant with the Agrobacterium of the present invention, gene targeting efficiency can be increased.

Preferably, in the Agrobacterium of the pesent invention, the function of a gene associated with T-DNA random insertion is lost or reduced. The term “T-DNA random insertion” is used in the present description to include all types of T-DNA insertions, other than the insertion by homologous recombination. Specific examples of the gene associated with such T-DNA random insertion include, but are not limited to, a Vir gene, a ChvA gene, a ChvB gene, a PscA gene, an Att gene, and a function-unknown gene (unknown gene).

Examples of the transcription unit of the Vir gene include virA, virB, virC1, virD3, virD4, virD5, virE1, virE1, virE2, virF, virG, virH1, and virK. Each transcriptional region comprises one or plural genes. The Vir gene is prefetably any one or mole selected from among virA, virC1, virD3, virD4, virD5, virE0, virE1, virE2, virF, virH1 and virK, more preferably, any one or more selected from among virC1 virD3, virD4, virD5, virE0, virE1, virE2, virF, virH1 and virK, further preferably, any one or more selected from among virE0, virE1, virE2, virF, virH1 and virK, and particularly preferably, any one or more selected from among virE1, VirE1 and virE2.

Specific examples of the Vir gene include an ATU6150(virH1) gene, an ATU6154 (virF) gene, an ATU6166(virA) gene, an ATU6183 (virD3) gene, an ATU6184 (virD4) gene, an ATU6185 (virD5) gene, an ATU6188 (virE0) gene, an ATU6189 (virE1) gene, an ATU6190 (virE2) gene, an ATU6191 (virE3) gene, an ATU6156 (virK) gene, and an ATU6180 (virC1)gene.

Among the above-described genes, the Vir gene is preferably any one or more selected from a TU6150 (virH1) gene, an ATU6154, (virF) gene, an ATU6183 (virD3) gene, an ATU6184 (virD4) gene, an ATU6183 (virD5) gene, an ATU6188 (virE0) gene, an ATU6189 (virE1) gene, an ATU61891 (vir E3) gene, an ATU6156 (virK) gene, and an ATU6180 (virC1) gene.

The Vir gene is more preferably any one or more selected from an ATU6191 (vir E3) gene, an ATU6150 (virH1) gene, an ATU6188 (virE0) gene, an ATU6154 (virF) gene, an an ATU6156 (virK) gene.

The Vir gene is further preferably any one or more selected from an ATU6154 (virF) gene, and an ATU6156 (virK) gene.

The Vir gene is particularly preferably any one or more selected from an ATU6191 (virE3) gene, and ATU6154 (virF) gene, and an ATU6156 (virK) gene.

Examples of the function-unknown gene (unknown gene) include an ATU3081 gene and an ATU4309 gene.

The number of genes whose function is to be lost or reduced is not particularly limited, as long as it is 1 or greater, and may be any number of 1 to 11. For example, the functions of 1 to 10 genes, and preferably of 1 to 5 genes can be lost or reduced.

The nucleotide sequences of an ATU3081 gene (SEQ ID NO: 1), an ATU4309 gene (SEQ ID NO: 2), an ATU6150 (virH1) gene (SEQ ID NO: 3), an ATU6154 (virF) gene (SEQ ID No. 4), an ATU6166 (virA) gene (SEQ ID NO. 5), an ATU6183 (virD3) gene (SEQ ID NO: 6), an ATU6184 (virD4) gene (SEQ ID NO: 7), an ATU6185 (virD5) gene (SEQ ID NO: 8), an ATU6188 (virE0) gene (SEQ ID NO: 9), an ATU6189 (virE1) gene (SEQ ID NO: 10), an ATU6190 (virE2) gene (SEQ ID NO: 11), an ATU6191 (virE3) gene (SEQ ID NO: 12), an ATU6156 (virK) gene (SEQ ID NO: 13) and an ATU6180 (virC1) gene (SEQ ID NO: 14) in the Agrobacterium A208 strain are shown in SEQ ID NOS: 1 to 14 in the sequence listing, respectively. The nucleotide sequences of an ATU6150 (virH1) gene (SEQ ID NO: 15), an ATU6154 (virF) gene (SEQ ID NO: 16), an ATU6166 (virA) gene (SEQ ID NO: 17), an ATU6183 (virD3) gene (SEQ ID NO: 18), an ATU6184 (virD4) gene (SEQ ID NO: 19), an ATU6185 (virD5) gene (SEQ ID NO: 20), an ATU16189 (virE 1) gene (SEQ ID NO: 21), an ATU6190 (virE2) gene (SEQ ID NO: 22), an ATU6191 (virE3) gene (SEQ ID NO: 23), an ATU6156 (yirK) gene (SEQ ID NO: 24) and an ATU6180 (virC1) gene (SEQ ID NO: 25) in the Agrobacterium EHA101 strain are shown in SEQ ID NOS: 15 to 25 in the sequence listing, respectively.

The Agrobacterium of the present invention does not only include an Agrobacterium, in which the functions of the aforementioned 14 types of genes are lost or reduced, but it also includes an Agrobacterium, in which the functions of homologous genes of the aforementioned genes are lost or reduced. The homologous gene means a gene, which consists of a nucleotide sequence having homology (which is also referred to as “sequence identity”) of 40% or more, preferably 50% or more, more preferably 60% or more, even more preferably 65% or more, further preferably 70% or more, further preferably 75% or more, further preferably 80% or more, still further preferably 85% or more, still further preferably 90% or more, and still further preferably 95% or more, with the nucleotide sequence of any one of an ATU3081 gene, an ATU4309 gene, an ATU6150 (virH1) gene, an ATU6154 (virF) gene, an ATU6166 (virA) gene, an ATU6183 (virD3) gene, an AVU6184 (virD4) gene, an ATU6185 (virD5) gene, an ATU6188 (virE0) gene, an ATU6189 (VirE1) gene, an ATU6190 (virE2) gene, an ATU6191 (virE3) gene, an ATU6156 (virK) gene, and an ATL6180 (virC1) gene, and which has functions equivalent to those of each of the above-described genes. The following Table 1 shows the gene sizes and homology levels of homologous genes in the Agrobacterium EHA101 strain, as several examples of the homologous genes.

TABLE 1

| | | Gene Size | Gene size | |
|---|---|---|---|---|
| | | (Agrobacterium | (Agrobacterium | Homology |
| Gene name | Accession | A208 strain) | EHA101 strain) | (DNA level) |
| — | ATU3081 | 1494 bp | 1494 bp | 100% |
| — | ATU4309 | 1032 bp | 1032 bp | 100% |
| virH1 | ATU6150 | 1260 bp | 1269 bp | 79% |
| virF | ATU6154 | 939 bp | 942 bp | 45% |
| virA | ATU6166 | 2502 bp | 2498 bp | 76% |
| virD3 | ATU6183 | 2022 bp | 639 bp | 49% |
| virD4 | ATU6184 | 2007 bp | 1959 bp | 78% |
| virD5 | ATU6185 | 2523 bp | 2511 bp | 69% |
| virE0 | ATU6188 | 252 bp | — | — |
| virE1 | ATU6189 | 192 bp | 198 bp | 70% |
| virE2 | ATU6190 | 1671 bp | 1650 bp | 72% |
| virE3 | ATU6191 | 2055 bp | 2019 bp | 69% |
| virK | ATU6156 | 435 bp | 438 bp | 75% |
| virC1 | ATU6180 | 696 bp | 696 bp | 81% |

The method of losing or reducing the function of the above described genes is not particularly limited. A mutation or deletion may be introduced into the nucleotide sequences of the above-described one or more genes, so as to lose or reduce the functions of the gene(s), or all of the above-described one or more genes may be deleted, so as to delete the functions of the gene(s). Methods of site-specifically mutatating a gene on the chromosome are well know to a person skilled in the art. Representative examples of such a method include a method of utilizing the mechanism of transposon and homologous recombination (Ohman et al., J. Bacteriol., 162: 1068-1074 (1985)) and a method involving, as principles, site-specific integration caused by the mechanism of homologous recombination and dropping caused by homologous recombination as a second stage (Noti et al., Methods Enzymol., 154: 197-217 (1987)). Moreover, there can also be utilized a method which comprises allowing a sacB gene derived from Bacillus subtilis to coexist with a concerned gene, and then easily isolating, as a sucrose resistance strain, a microorganism strain in winch the concerned gene has been dropped by the second-stage homologous recombination (Schweizer, Mol. Microbiol., 6: 1195-1204 (1992), Lenz et al., J. Bacteriol., 176: 4385-4393 (1994)). However, the method is not particularly limited. Preferably, a get associated with T-DNA random insertion, which is in the chromosome of an Agrobacterium, can be disrupted according to a homologous recombination method.

In addition, the present invention relates to a method for producing a transformed plant, which comprises infecting plant cells with an Agrobacterium having a T-DNA nuclear translocation ability equivalent to that of a wild-type Agrobacterium and also having a T-DNA chromosomal insertion ability that is lost or reduced in comparison to a wild-type Agrobacterium wherein the Agrobacterium has a targeting vector comprising a foreign gene.

The “plant transformation method that involves mediation of Agrobacterium,” which becomes a base of the method of the present invention, is generally referred to as an “Agrobacterium-mediated transformation method” or an “Agrobacterium method”. This is a method of transforming a plant by introducing a foreign gene into the genome of a plant cell through the mediation of Agrobacterium. Such Agrobacterium inserts a T-DNA region contained in a vector, such as a plasmid, possessed by the Agrobacterium itself, into the plant chromosomal DNA, in the plant cells infected with the Agrobacterium. Thus, according to the plant transformation method involving the mediation of Agrobacterium, under the control of a promoter and a terminator present between the right border sequence (RB) and the left border sequence (LB) of a T-DNA region, a vector (preferably, at binary vector), into which a foreign gene to be introduced into a plant has been incorporated, is introduced into Agrobacterium according to an ordinary method, and then, the Agrobacterium is inoculated into the plant, and the plant is thereby infected therewith, so that the foreign gene in the T-DNA region can be introduced into the plant.

More specifically, for example, in the method of the present invention, a transformation method, which comprises infecting the callus or tissue section of a plant in an in vitro culture system with Agrobacterium, so that a foreign gene is introduced therein, and then regenerating the foreign gene in the plant body by in vitro culture, so as to produce a transformed plant body, may be used as a plant transformation method involving the mediation of Agrobacterium. Alternatively, in the method of the present invention, an in planta transformation method, which is generally used Arabidopsis thaliana and is called “Floral dip method,” may also be used. Furthermore, are the method of the present invention, the in planta transformation method comprising inoculating a foreign gene-containing vector into Agrobacterium and then infecting the meristem of an individual plant (a plant body or a seed) with the Agrobacterium can be preferably used as a plant transformation method involving the mediation ofAgrobacterium.

In one embodiment, in the method of the present invention, a plant can also be transformed according to a plant transformation method involving the mediation of Agrobacterium, particularly, using Agrobacterium retaining a vector having a T-DNA region that contains foreign gene but does not contain a selection marker gene, namely, a vector into the T-DNA region of which a foreign gene has been introduced (a foreign gene-containing vector) but a selection marker gene has not been inserted therein. The “foreign gene” in the T-DNA region used herein is not a selection marker gene. The definition, specific examples and the like of the term “selection marker gene” will be described later. In this method of the present invention, a selection marker gene usually used for selection transformant is not introduced, together with a foreign gene, into host plant cells, and a foreign gene introduced into the genome, or a nucleic acid such as mRNA or a protein expressed from the gene, is detected, so that a plant transformant may be selected. Since this plant transformant does not contain a selection marker gene, it cannot be selected by a selection method using a selection marker (for example, selection based on antibiotic resistance or herbicide resistance). In this method, by using the method in combination with an Agrobacterium transformation method having high transformation efficiency (for example, the after-mentioned method), a transformant can be efficiently obtained without introducing a selection marker gene into the genome of a host plant.

In the present invention by using an Agrobacterium strain in which a specific gene is disrupted, homologous recombination efficiency can be increased. This is considered because insertion of T-DNA into plant genome is suppressed by deletion of a specific gene, and as a result, homologous recombination is promoted.

A bacterial suspension of Agrobacterium to be inoculate into a plant may further comprise a surfactant such as Tween 20. Such a bacterial suspension of Agrobacterium may also comprise phenols such as acetosyringone. These components are able to further improve transformation efficiency.

The plant transformation method of the present invention can be applied to any given plants that be infected with Agrobacterium. The target, to which the plant transformation method of the present invention can be applied, may be either a dicotyledon or a monocotyledon. The target plant, to which the present plant transformation method can be applied, is not particularly limited. Examples ofthe target plant include plants belonging to: Poaceae[wheat (Triticum aestivum L.) rice (Oryza sativa), barley (Hordeum vulgare L.), corn (Zea mays L.), sorghum (Sorghum bicolor (L.) Moench), erianthus (Erianthus spp), Guinea grass (Panicum maximum Jacq.), miscanthus (Miscanthus spp), sugarcane (Saccharum officinarum L.) napier grass (Pennisetum purpureum Schumach), pampas grass (Cortaderia argentea Stapf), Perennial ryearass (Lolium perenne L.), Italian ryearass (Lolium multiflorum Lam.), meadow fescue (Festuca pratensis Huds.), tall fescue (Festuca arundinacea Schreb.), Orchard grass (Dactylis glomerata L.), timothy (Phleum protease L.), etc.]; Leguminosae [soybean (Glycine max), adzuki bean (Vigna angularis Willd.), kidney bean (Phaseolus vulgaris L.), fava bean (Vices faba L.), etc.]; Malvaceae [cotton (Gossypium spp.), kenaf (Hibiscus cannabinus), okra (Abelmoschus esculentus), etc.]; Solanaceae [eggplant (Solanum melongena L.), tomato (Solanum lycopersicum), green peper (Capsicum annuum L. var. angulosum Mill.). capsicum (Capsicum annuum L.), tobacco (Nicotiana tabacum L.), etc. ]; Brassicaceae [Arabidopsis (Arabidopsis thaliana) rape (Brassica campestris L.), Chinese cabbage (Brassica pekinensis Rupr.), cabbage (Brassica oleracea L. var.capitata L.), radish (Raphanus sativus L.), rape seed (Brassica campestris L., B. napus L.), etc.]; Cucurbitaceae [cucumber (Cucumis sativus L.), melon (Cucumis melo L.), watermelon (Citrullus vulgars Schrad.), pumpkin (C. moschata Duch., C. maxima Duch.), etc.]; Convolvulacea [sweet potato (Ipomoea batatas), etc.], Liliaceae [spring onion (Allium fistulosum L.), onion (Allium cepa L.), Chinese chive (Allium tuberosum Rottl.), garlic (Allium sativum L.), asparagus (Asparatus officinalis L.), etc.]; Labiatae [Japanese basil (Perilla frutescens Britt. var. crispa), etc.]; Asteraceae [chrysanthemum (Chrysanthemum morifolium), garland chrysanthemum (Chrysanthemum coronanum L.) lettuce (Lactuca sativa L. var. capitata L.) etc.]; Rosaceae [rose (Rose hybrida Hort.), strawberry (Fragaria x ananassa Duch.), etc.]; Rutaceae [orange (Citras unshiu), Japanese pepper (Zanthoxylum piperitum DC.), etc.]; Myrtaceae [Eucalyptus (Eucalyptus globulus Labill), etc.], Salicaceae [poplar (Populas nigra L. var. italica Koehne), etc.]; Chenopodiaceae [spinach (Spinacia oleracea L.), sugar beet (Beta vulgaris L.), etc.]; Gentianaceae [gentian (Gentiana scabra Bunge var. buergeri Maxim.),etc.]; and Caryophyllaceae [carnation (Dianthus caryophyllus L.), etc.]. Plants, which have provided only low gene transfer efficiency by the conventional transformation methods, such as poaceous, leguminous and malvaceous plants, are particularly preferable as target plants, to which, the transformation method of the present invention is applied.

The Agrobacterium used in the method of the present invention is not particularly limited, as long as it is a plant pathogen belonging to the genus Rhizobium, capable of causing transformation mediated by Agrobacterium. Examples of such Agrobacterium include Agrobacterium tumefaciens, Agrobacterium vitis, Agrobacterium rhizogenes, and Agrobacterium radiobacter. Specific examples of Agrobacterium include, but are not limited to, Agrobacterium tumefaciens LBA4404 strain, C58 strain, EHA101 strain, A208 strain, Agrobacterium vitis F2/5 strain, S4 strain, Agobacterium rltizogenes A4 strain, LBA9402 strain, and the derived strains thereof.

A foreign gene to be introduced into a plant is introduced into a T-DNA region of a vector comprising the T-DNA region to produce a foreign gene-containing vector i.e., a targeting vector having a foreign gene), and the thus produced vector is then introduced into Agrobacterium, thereby producing an Agrobacterium having a foreign gene-containing vector. The vector comprising the T-DNA region comprises a T-DNA derived from an Agrobacterium plasmid, namely, of nucleic acid sequence sandwiched between a right border sequence (RB) and a left bolder sequence (LB), and a replication origin, and this is a vector autonomously replicating in Agrobacterium. The T-DNA region in the vector preferably comprises a promoter and a terminator between the RB and LB sequences. The vector comprising the T-DNA region is more preferably a binary vecor, which comprises the repication origins of other microorganisms such as E. coli yeast, and is capable of autonomously replicating also in those microorganisms. The vector comprising the T-DNA region may comprise a vir gene outside of the T-DNA region. As vectors comprising a T-DNA region for plant transformation, which are preferably used for introduction of a foreign gene into a plant, multiple types of vectors are commerially available. Examples of the vector comprising the T-DNA region include, but are not limited to, pIG121-Hm (Ohta, S., et al., Plant Cell Physiol. 31, 805-813, 1990), pCAMBIA (Marker Gene Technologies, Inc.), pR1909 (TaKaRa), pRI101 (TaKaRa), pBI (Inplanta Innovations Inc.), pBIN (Bevan, M. et al., Nucleic Acids Res. 12. 8711-8721. 1984), pPZP (Hajdukiewicz, P. et al., Plant Mol. Biol, 25, 989-994 1994), pGreen (Hellens, R. et al., Plant Mol. Biol. 42, 819-832. 2000), pBIBAC (Liu. et al., Proc. Natl. Acad. Sci. USA 96, 6535-6540. 1999), pGA, SEV, pEND4K, pC1B10, pMRK63, pGPTV, pCGN1547, pART, pGKB5, pMJD80, pMJD81, pBINPLUS, pRT100, pCB, pMDC, pRCS2, and pORE.

In a preferred embodiment of the present invention, a vector into the T-DNA region of which a foreign gene has been introduced (a foreign gene-containing vector) may comprise a selection marker gene outside of the T-DNA region. However, it is preferable that such a selection marker gene be not comprised in the T-DNA region. The selection marker gene is used in the present invention to mean a gene imparting ability only to transformed cells under predetermined conditions, and enabling selection of the transformed cells, ora fluorescent protein gene or an enzyme gene catalyzing a coloring reaction. Representative examples of the selection marker gene include a drug resistance gene and a nutrient-requiring complementary gene. Specific examples of the selection marker gene include, but are not limited to, a kanamycin resistance gene, a hygromycin resistance gene, a phosphinothricin resistance gene, a bialaphos resistance gene, a gentamicin resistance gene, a sulfonylurea resistance gene, a dihydrofolate reductase gene, a bleomycin resistance gene, a luciferase gene, a β-galactosidase gene, a β-glucuronidase gene, and a green fluorescence protein (GFP) gene. Since the foreign gene-containing vector does not comprise a selection marker gene in the T-DNA region thereof, the selection marker gene is not ntroduced into a transformed plant, so that a transformed plant with higher trait stability and higher safety can be obtained. Since transformation efficiency is significantly increased in the method of the present invention, a transformed plant can be selected with sufficient efficiency, without introduction of a selection marker gene, and thus, without using a change in the phenotype caused by the expression of the selection marker gene as an indicator.

A foreign gene to be comprised in a vector comprising a T-DNA reaion is any given gene intended to induce expression in a plant, and it may be either a plant-derived gene or an animal-derived gene. The “foreign gene” indicates a nucleic acid (generally, DNA) introduced from the outside through the mediation of Agrobacterium, and it may be, for example, a gene that may be isolated from a host plant into which the gene is to be introduced, or from an organism species or strain of the same species as the host plant. The “foreign gene” of the present invention may encode a protein or may also encode functional RNA. Examples of the foreign gene include a group of genes, which are associated with seed yield increase, environmental stress (low temperature, drying, salts, virus, disease, and high temperature) resistance, photosynthetic function enhancement, biomass production, useful substance production, etc., but examples are not limited thereto.

Introduction of a foreign gene-containing vector into Agrobacterium can be carried out according to an ordinary method. For example, a foreign gene-containing vector can be introduced into Agrobacterium according to a freezing-thawing method or a particle gun method. To explain an example of the freezing-thawing method just briefly, competent cells of Agrobacterium are mixed with a foreign gene-containing vector, the obtained mixture is then incubated on ice for 5 minutes, and it is then frozen in liquid nitrogen for 5 minutes. Subsequently, the resultant is incubated at 37° C. for 5 minutes, so that it is thawed, and it is then subjected to a shaking culture at room temperature or at 2° C. for to 2 to 4 hours. Thereafter, the reaction culture is cultured in an antibiotic-containing medium, and the formed single clones may be collected.

In the present invention, plant cells can be infected with an Agrobacterium. A transformed plant can be produced by infecting plant cells with an Agrobacterium having a foreign gene-containing vector.

Inoculation of Agrobacterium may be carried out according to an ordinary method. In the present invention, in the case of using an in planta transformation method, an Agrobacterium having a foreign gene-containing vector is preferably inoculated into the meristem of an individual plant (a plant body or a seed). Any given meristem of an individual plant can be used as an inoculation site. Such an Agrobacterium is preferably inoculated into, for example, the meristem of the shoot apex or axillary bud of a juvenile plant or seedling, or the meristem of the embryo of a seed. In the case of a poaceous plant for example, an Agrobacterium is preferably inoculated into the meristem of the embryo of a seed. Inoculation of an Agrobacterium into the meristem of such a seed embryo may be inoculation of an Agrobacterium into a shoot (stem and leave) germinating from a seed, an embryonic portion around a shoot base portion, or a root.

Inoculation of Agrobacterium into the meristem is preferably carrried out in a wound site on the meristem. Such a wound site may be created, for example, by pricking several sites on the meristem with a sterilized needle (for example, with a diameter of 0.71 mm) to make stab wounds. The size of such stab wounds may be approximately 0.5 mm to 2 mm, but it is not limited thereto. Otherwise, other types of wounds, such as small incised wounds, may also be created on the meristem. After creation of wounds, Agrobacterium may be inoculated into such wound sites, or after inoculation of Agrobacterium into a plant, wounds may be created on the inoculation sites.

In order to promote infection with Agrobacterium, a plant, into which such Agrobacterium has been inoculated, may be co-cultured with Agrobacterium. In the method of the present invention, the co-culture is preferably carried out at a temperature of 23° C. or higher, preferably at 25° C. to 30° C., and more preferably at 28° C. The co-culture may be carried out for an ordinary period of time. For example, the co-culture is preferably carried out, for example, for 12 hours to 10 days, preferably for 24 hours, to 5 days, and more preferably for 36 hours to 4 days. By performing a co-culture in such a temperature range, the growth of Agrobacterium can be promoted, and thus, infection can also be promoted.

After completion of the infection, a plant into which Agrobacterium has been inoculated may be subjected to a treatment of eliminating the Agrobacterium. The Agrobacterium elimination treatment can be carried out by treating the Agrobacterium with an antibiotic such as cefotaxime.

A plant, which has been infected with Agrobacterium and then, as necessary, has been disinfected, is allowed to grow under suitable cultivation conditions. At a time point in which the plant has reached a predetermined growing stage, it is preferably confirmed by genomic PCR or the like that the introduced foreign gene has been incorporated into the genome. The plant, in which introduction of the foreign gene has been confirmed, is selected as a transformed plant, and this plant is defined as T0 generation.

The plant of TO generation s allcm d its form a flower bud, and is trey allowed to bleed, so that the plant forms seeds. The thus obtained plant is a plant of T1 generation. Even in the case of the plant of T1 generation, a stable transformed plant can be selected and obtained by confining incorporation of a foreign gene into the genome.

With regard to the thus obtained transformed plant, gene targeting efficiency can be calculated. Such gene targeting efficiency can be calculated according to the expression: homologous recombination efficiency/random recombination efficiency. Herein, the term “homologous recombination efficiency” is used to mean the number of plants undergoing homologous recombination/the number of plants subjected to transformation, whereas the term “random recombination efficiency” is used to mean the number of plants undergoing random recombination/the number of plants subjected to transformation.

The homologous recombination efficiency obtain upon transformation of a plant with the Agrobacterium of the present invention is increased, preferably 2 time or more, more preferably 5 time or more, even more preferably 10 time or more, further preferably 15 time or more, and particularly preferably 20 time or more, in comparison to that of a wild-type Agrobacterium. The higher the aforementioned improvement magnification, the more preferable it is. The upper limit is not particularly limited, and it is generally 200 time or less, and for example, 100 time or less.

The gene targeting efficiency obtained upon transformation of a plant with the Agrobacterium of the present invention is increased, preferably 2 time or more, more preferably 4 time or more, even more preferably 10 time or more, further preferably 15 time or more, and particularly preferably 20 time or more, in comparison to that of a wild-type Agrobacterium. The higher the aforementioned improvement magnification, the more preferable it is. The upper limit is not particularly limited, and it is generally 200 time or less, and for example, 100 time or less.

The gene targeting efficiency obtained upon transformation with the Agrobacterium of the present invention is preferably 1% or more, 1.5% or more, even more preferably 5% or more, further preferably 8% or more, and particularly preferably 10% or more. The higher the gene targeting efficiency, the more preferable it is. The upper limit is not particularly limited, and it is generally 70% or less, and for example, 50% or less.

### EXAMPLES

Hereinafter, the present invention will be more specifically described in the following examples. However, these examples are not intended to limit the technical scope of the present invention.

### Example 1

### Production of Gene Disrupted Strain of Agrobacterium

Gene disrupted strains of Agrobacterium A208 (C58 chromosome, nopaline-type T37pTi) (Wirawan IG, Kang HW, Kojima M (1993) Isolation and characterization of a new chromosomal virulence gene of Agrobacterium tumefaciens, J Bateriol 175: 3208-3212) were produced by applying a “method comprising, as principles, site-specific incorporation caused by the mechanism of homologous recombination and dropping by homologous recombination as a second stage,” which is described in JP Patent Publication (Kokai) No. 2007-259708 A.

Primers used for production of gene disrupted strains of Agrobacterium will be shown below.

| | |
|---|---|
| ATU3081 Forward1 | TTAGCGCCTCTGAAATTCGG (SEQ ID NO: 26) |
| ATU3081 Reverse1 | AAGGCACTTCCTCAGCGACCA (SEQ ID NO: 27) |
| ATU3081 Forward2 | TTACTTCTGACCGCAAGAGATC (SEQ ID NO: 28) |
| ATU3081 Reverse2 | TTCGCCAGATGCTCAGCGAT (SEQ ID NO: 29) |
| ATU4309 Forward1 | GGTGGTGGAAAGCTCATGTC (SEQ ID NO: 30) |
| ATU4309 Reverse1 | CGATCCAGAAAATCCTGGCG (SEQ ID NO: 31) |
| ATU4309 Forward2 | CACCGGCTTCTGCGACGCGC (SEQ ID NO: 32) |
| ATU4309 Reverse2 | AATCTATCCGTAGATCATCCTC (SEQ ID NO: 33) |
| ATU6150 Forward1 | GTGGATATCGAGGGGACAC (SEQ ID NO: 34) |
| ATU6150 Reverse1 | ACATACTTTCATCGAAGTCG (SEQ ID NO: 35) |
| ATU6150 Forward2 | GGTCACCGTTCCGTTCGGTT (SEQ ID NO: 36) |
| ATU6150 Reverse2 | TCTCCGCGAACAGATCTACTG (SEQ ID NO: 37) |
| ATU6154 Forward1 | CAATTGACTGACCTCGCGAA (SEQ ID NO: 38) |
| ATU6154 Reverse1 | CGTGCATGCTCCTTCTTTCT (SEQ ID NO: 39) |
| ATU6154 Forward2 | TTCCTGGATCCACCGCGCTG (SEQ ID NO: 40) |
| ATU6154 Reverse2 | ATCGGCTCAATTGATATCCC (SEQ ID NO: 41) |
| ATU6166 Forward1 | TTTGCTTGGTCACCTGAAAC (SEQ ID NO: 42) |
| ATU6166 Reverse1 | CCGCACTTACGTCCTCGTAC (SEQ ID NO: 43) |
| ATU6166 Forward2 | AGTTGAGGATGTTTTTCAGGAG (SEQ ID NO: 44) |
| ATU6166 Reverse2 | TAGGGTCGGTTACTCTAGGAT (SEQ ID NO: 45) |
| ATU6183 Forward1 | AAACCGCACTCCCGATATTC (SEQ ID NO: 46) |
| ATU6183 Reverse1 | CACTCGGTCCTTCTCTATCT (SEQ ID NO: 47) |
| ATU6183 Forward2 | AGGCTGTGTTGTTCGCAGCA (SEQ ID NO: 48) |
| ATU6183 Reverse2 | CCAAAGATTGGCCCCTTGATAC (SEQ ID NO: 49) |
| ATU6184 Forward1 | GTTGGTTCCCAATGCCAATC (SEQ ID NO: 50) |
| ATU6184 Reverse1 | CACTTCACCGAGATTCTTCG (SEQ ID NO: 51) |
| ATU6184 Forward2 | CTTCAAGCTGCCTTTCACAT (SEQ ID NO: 52) |
| ATU6184 Reverse2 | AAGATCAAAGGCGACTCCTC (SEQ ID NO: 53) |
| ATU6185 Forward1 | GCCTTTCACATTGGAATCAT (SEQ ID NO: 54) |
| ATU6185 Reverse1 | ATGGGAAGACCTCGTTGTCA (SEQ ID NO: 55) |
| ATU6185 Forward2 | AAGGTCGTCCACGATACTTT (SEQ ID NO: 56) |
| ATU6185 Reverse2 | GGCACTGCTGTCAAGAAATC (SEQ ID NO: 57) |
| ATU6188 Forward1 | ATTCCTGAGCATTGAGGTCC (SEQ ID NO: 58) |
| ATU6188 Reverse1 | TGCTTTCTAGGCTGCTGCAG (SEQ ID NO: 59) |
| ATU6188 Forward2 | ATGGAGCAAACCTTAATTTG (SEQ ID NO: 60) |
| ATU6188 Reverse2 | GGACATCCCGGTGGAATTTA (SEQ ID NO: 61) |
| ATU6189 Forward1 | ACCGAAGGCTCAATTCTATT (SEQ ID NO: 62) |
| ART6189 Reverse1 | ATCATTGTTTCTCCTACAGA (SEQ ID NO: 63) |
| ATU6189 Forward2 | AAGGAGTTAGACGATGGATC (SEQ ID NO: 64) |
| ATU6189 Reverse2 | CAGCTCGCATTGAATTCCG (SEQ ID NO: 65) |
| ATU6190 Forward1 | ACCTGGCGGCAAACCGACA (SEQ ID NO: 66) |
| ATU6190 Reverse1 | TAGCCGGCTAGGTTTTCTTC (SEQ ID NO: 67) |
| ATU6190 Forward2 | GGTCGACACGACGAAGAAG (SEQ ID NO: 68) |
| ATU6190 Reverse2 | TATTTCACATGCTTCGCGCG (SEQ ID NO: 69) |
| ATU6191 Forward1 | ACCGCGTCAGTGACGAAGCC (SEQ ID NO: 70) |
| ATU6191 Reverse1 | GCGTTACGCTTCAGAACCTT (SEQ ID NO: 71) |
| ATU6191 Forward2 | TGAAACAGGCCAAGATGTG (SEQ ID NO: 72) |
| ATU6191 Reverse2 | TCATTCACCATCGCGCGCCA (SEQ ID NO: 73) |
| ATU6156 Forward1 | ATCAATTGAGCCGATCTTCC (SEQ ID NO: 74) |
| ATU6156 Reverse1 | AGGTGATCCTATTTAGATTG (SEQ ID NO: 75) |
| ATU6156 Forward2 | TGATCTCTGACTCCTGTGT (SEQ ID NO: 76) |
| ATU6156 Reverse2 | CTTCCATGTGCTGATTTCCA (SEQ ID NO: 77) |
| ATU6180 Forward1 | TCTCTGCGTCGATTTCAAGA (SEQ ID NO: 78) |
| ATU6180 Reverse1 | TCCTTATCCTGTCGATTTTG (SEQ ID NO: 79) |
| ATU6180 Forward2 | CAGATGGGAATTCGCAAACC (SEQ ID NO: 80) |
| ATU6180 Reverse2 | TGTGTCTAGGCTATCGGATAAC (SEQ ID NO: 81) |

Moreover, gene disruption was confirmed by performing genomic PCR on the produced Agrobacterium strains. Conditions for the PCR reaction are shown in the following Table 2.

TABLE 2

| | | | Number of |
|---|---|---|---|
| Step | ° C. | Time | cycles |
| 1 | 94 | 3 min | 1 |
| 2 | 94 | 30 sec | 35 |
| 3 | 57 | 30 sec | |
| 4 | 72 | 3 min | |
| 5 | 72 | 5 min | 1 |
| 6 | 4 | ∞ | |

In the genomic PCR performed to confirm gene disruption, the following primers were used.

| | |
|---|---|
| ATU3081 Forward1 | TTAGCGCCTCTGAAATTCGG (SEQ ID NO: 26) |
| ATU3081 Reverse2 | TTCGCCAGATGCTCAGCGAT (SEQ ID NO: 29) |
| ATU4309 Forward1 | GGTGGTTGGAAAGCTCATGTC (SEQ ID NO: 30) |
| ATU4309 Reverse2 | AATCTATCCGTAGATCATCCTA (SEQ ID NO: 33) |
| ATU6150 Forward1 | GTGGATATCGAGGGGACAC (SEQ ID NO: 34) |
| ATU6150 Reverse2 | TCTCCGCGAACAGATCTACTG (SEQ ID NO: 37) |
| ATU6154 Forward1 | CAATTGACTGACCTCGCGAA (SEQ ID NO: 38) |
| ATU6154 Reverse2 | ATUGGCTCAATTGATATCCC (SEQ ID NO: 41) |
| ATU6166 Forward1 | TTTGCTTGGTCACCTGAAAC (SEQ ID NO: 42) |
| ATU6166 Reverse2 | TAGGGTCGGTTACTCTAGGAT (SEQ ID NO: 45) |
| ATU6183 Forward1 | AAACCGCACTCCCGATATTC (SEQ ID NO: 46) |
| ATU6183 Reverse2 | CCAAAGATTGGCCCCTTGATAC (SEQ ID NO: 49) |
| ATU6184 Forward1 | GTTGGTTCCCAATGCCAATC (SEQ ID NO: 50) |
| ATU6184 Reverse2 | AAGATCAAAGGCGACTCCTC (SEQ ID NO: 53) |
| ATU6185 Forward1 | GCCTTTCACATTGGAATCAT (SEQ ID NO: 54) |
| ATU6185 Reverse2 | GGCACTGTCTGTCAAGAAATC (SEQ ID NO: 57) |
| ATU6188 Forward1 | ATTCCTGAGCATTGAGGTCC (SEQ ID NO: 58) |
| ATU6188 Reverse2 | GGACATCCCGGTGGAATTTA (SEQ ID NO: 61) |
| ATU6189 Forward1 | ACCGAAGGCTCAATTCTATT (SEQ ID NO: 62) |
| ATU6189 Reverse2 | CAGCTCGCATTGAATTCCG (SEQ ID NO: 65) |
| ATU6190 Forward1 | ACCTGGCGGCAAACCGACA (SEQ ID NO: 66) |
| ATU6190 Reverse2 | TATTTCACATGCTTCGCGCG (SEQ ID NO: 69) |
| ATU6191 Forward1 | ACCGCGTCAGTGACGAAGCC (SEQ ID NO: 70) |
| ATU6191 Reverse2 | TCATTCACCATCGCGCGCCA (SEQ ID NO: 73) |
| ATU6156 Forward1 | ATCAATTGAGCCGATCTTCC (SEQ ID NO: 74) |
| ATU6156 Reverse2 | CTTCCATGTGCTGATTTCCA (SEQ ID NO: 77) |
| ATU6180 Forward1 | TCTCTGCGTCGATTTCAAGA (SEQ ID NO: 78) |
| ATU6180 Reverse2 | TGTGTGAGGGATCGGATAAC (SEQ ID NO: 81) |

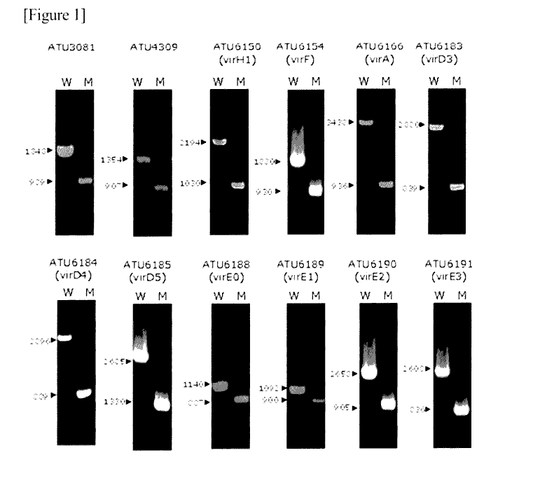

As a result of the genomic PCR, it was confirmed that a gene of interest was disrupted in each of the Agrobacterium gene disrupted strains (FIG. 1). In addition, a sequencing analysis was carried out on each disrupted strain, and it was confirmed that the gene was disrupted at a DNA sequence level.

### Example 2

### Confirmation of Pathogenicity (T-DNA Chromosomal Insertion Ability) and T-DNA Nuclear Translocation Ability of Agrobacterium Gene Disrupted Strains

The pathogenicity (T-DNA chromosomal instertion ability) of the Agrobacterium gene disrupted strains was confirmed by the following method. The Agrobacterium gene disrupted strains produced in [Example 1] were each cultured in an LB solid medium (Difco) at 25° C. for 48 hours, and the cultured cells were then inoculated into a leaf of a plant body of kalanchoe (Kalanchoe daigremontiana). The presence or absence of pathogenicity (T-DNA chromosomal insertion ability) was determined three weeks after the inoculation, based on the presence or absence of formation of a tumor (crown gall) in the inoculation site. Moreover, the size of the tumor was compared with that of a wild-type Agrobacterium strain, and the strength of pathogenicity was evaluated.

Moreover, T-DNA nuclear translocation ability was confirmed based on the transient expression of a GUS gene. The method will be described below. Strains were prepared by introducing a plasmid vector pIg121-Hm into the Agrobacterium gene disrupted strains produced in [Example 1], and they were then cultured in an LB solid medium (Difco) at 25° C. for 48 hour. The cultured cells were then inoculated into a leaf of a plant body of kalanchoe (Kalanchoe daigremontiana). Two weeks after the inoculation, GUS activity was measured. Such GUS activity was measured by the method described in Journal of Bioscience and Bioengineering Vol. 90, 328-331, 2000.

The measurement results of the pathogenicity (T-DNA chromosomal insertion ability) and T-DNA nuclear translocation ability of the Agrobacterium gene disrupted strains are shown in Table 3. Evaluation standards of pathogenicity (T-DNA chromosomal insertion ability) and T-DNA nuclear translocation ability are as follows.

### Pathogenicity (T-DNA Chromosomal Insertion Ability):

++: Tumor size is equivalent to that of the wild type (A208) (size: 0.5 times or greater) (T-DNA chromosomal insertion ability is “strong”)
+: Tumor size is less than half of the wild type (A208) (T-DNA chromosomal insertion ability is “weak”)
−: Tumor cannot be confirmed at all by visual observation (T-DNA chromosomal insertion ability “lacked”)

### T-DNA Nuclear Translocation Ability:

++: with T-DNA nuclear translocation ability (GUS activity value is 100 [pmol of 4-MU/min/mg protein]or more)
+: with T-DNA nuclear translocation ability (GUS activity value is 20 to 100 [pmol of 4-Mu/min/mg protein])
−: without T-DNA nuclear translocation ability (GUS activity value is 20 [pmol of 4-MU/min/mg protein] or less)

TABLE 3

| Agrobacterium | T-DNA nuclear | T-DNA |
|---|---|---|
| endogenous gene to be | translocation | chromosomal |
| disrupted | ability | insertion ability |
| Agrobacterium wild-type | ++ | ++ |
| strain (control) | | |
| ATU3081 | ++ | − |
| ATU6154 (virF) | ++ | + |
| ATU6156 (virK) | ++ | − |
| ATU6166 (virA) | + | − |
| ATU6185 (virD5) | ++ | − |
| ATU6190 (virE2) | + | − |
| ATU6184 (virD4) | + | − |
| ATU6189 (virE1) | ++ | − |
| ATU6191 (virE3) | ++ | + |
| ATU6150 (virH1) | ++ | + |
| ATU6180 (virC1) | ++ | + |
| ATU6183 (virD3) | ++ | + |
| ATU6188 (virE0) | ++ | + |
| ATU4309 | ++ | + |

### EXAMPLE 3

### Construction of Gene Targeting (Homologous Recombination) Evaluation System Using Arabidopsis Thaliana

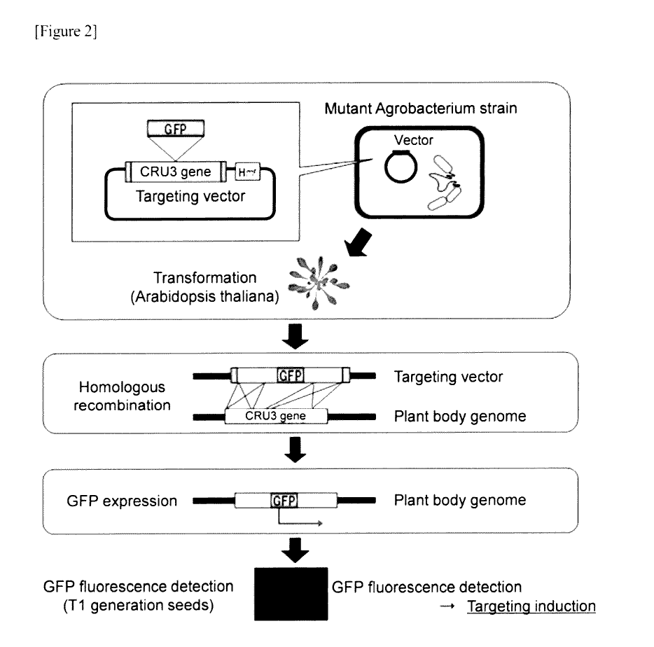

Cruciferin 3 thereinafter referred to as “CRU3”) that is a seed storage protein of Arabidopsis thaliana was used to produce a targeting reporter gene, and a gene targeting evaluation system was then constructed in Arabidopsis thaliana which is a dicotyledon (FIG. 2). Since a GFP fluorescence protein (GFP) has been inserted into a CRU3 gene comprised in a targeting vector to be used, when gene targeting is successfully carried out, specific GFP fluorescence is considered to be emitted in the endosperm that is an expression site of the CRU3 gene.

Moreover, in order to confirm the presence or absence of targeting by genomic PCR, the following primers and the PCR reaction conditions shown in Table 4 were used.
Forward primer: ctacgcgcatgaagatcaag (SEQ ID NO: 82)
Reverse primer: tcctcgcccttgctcaccat (SEQ ID NO: 83)

TABLE 4

| | | | Number of |
|---|---|---|---|
| Step | ° C. | Time | cycles |
| 1 | 94 | 3 min | 1 |
| 2 | 94 | 30 sec | 35 |
| 3 | 55 | 30 sec | |
| 4 | 72 | 3 min | |
| 5 | 72 | 5 min | 1 |
| 6 | 4 | ∞ | |

### EXAMPLE 4

### Confirmation of Effect of Improving Gene Targeting, Using Arabidopsis Thaliana (ATU6191Gene Disrupted Strain)

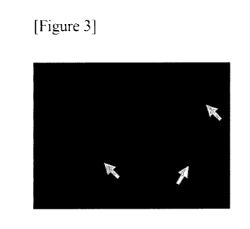

Arabidopsis thaliana was transformed using a mutant Agrobacterium strain (ATU6191 gene disrupted strain) into which the targeting vector produced in [Example 3] had been introduced, and the obtained T1 seeds were then recovered. Thereafter, T1 seeds, which emitted GFP fluorescence, were selected by microscopic observation (FIG. 3). T1 seeds, which did not emit GFP fluorescence, were germinated on a hygromycin selection medium, and it was considered that, in hygromycin resistant plants, random insertion took place by non-homologous recombination.

Among 5198 T1 seeds obtained after the transformation treatment, GFP fluorescence was observed in 3 seeds (3/5198=0.06%). In addition, as a result of the genomic PCR, fragments obtained only when gene targeting took place were confirmed in these seeds.

Moreover, the percentage of the occurrence of random insertion in the obtained 5198 T1 seeds by non-homologous recombination was calculated, using hygromycin resistance as an indicator. As a result, the percentage was found to be 1.05% (45/4280). The gene targeting efficiency (homologous recombination efficiency/random insertion efficiency) of the present mutant Agrobacterium strain was calculated to be 5.5%, based on the above results.

### Example 5

### Confirmation of Effect of Gene Targeting, Using Arabidopsis Thaliana (ATU6150 Gene Disrupted Strain, ATU6188 Gene Disrupted Strain, ATU6154 Gene Disrupted Strain, ATU3081 Gene Disrupted Strain, and ATU6156 Gene Disrupted Strain)

The effect of improving targeting was confirmed by the same method as that in [Example 4],with the exceptionn that the ATU6150 gene disrupted strain, the ATU6188 gene disrupted strain, the ATU6154 gene disrupted strain, the ATU3081 gene disrupted strain, or the ATU6156 gene disrupted strain was used as a mutant Agrobacterium strain. As a result, in all of the aforementioned five types of strains, the percentage seed, in which GFP fluorescence was observed, was 0.1% or more.

### Comparative Example 1

The effect of improving gene targeting was confirmed by the same method as that in [Example 4], with the exception that a wild-type Agrobacterium strain was used. As a result, no GFP fluorescence seeds could be found in 5000 T1 seeds obtained after completion of the transformation.

1. An Agrobacterium, which has a T-DNA nuclear translocation ability equivalent to a T-DNA nuclear translocation ability of a wild-type Agrobacterium, and does not have a T-DNA chromosomal insertion ability or has a reduced T-DNA chromosomal insertion ability compared to a T-DNA chromosomal insertion ability of the wild-type Agrobacterium.

2. The Agrobacterium according to claim 1, wherein the Agrobacterium has a homologous recombination efficiency of two or more times as large as a homologous recombination efficiency of the wild-type Agrobacterium, wherein the homologous recombination efficiency means a number of plants undergoing homologous recombination divided by a number of plants subjected to transformation.

3. The Agrobacterium according to claim 1, wherein the Agrobacterium has a gene targeting efficiency of two or more times as large as a gene targeting efficiency of the wild-type Agrobacterium, wherein the gene targeting efficiency means a ratio of homologous recombination efficiency to random recombination efficiency, the homologous recombination efficiency means a number of plants undergoing homologous recombination divided by a number of plants subjected to transformation, and the random recombination efficiency means a number of plants undergoing random recombination divided by a number of plants subjected to transformation.

4. The Agrobacterium according to claim 1, wherein the Agrobacterium has a gene targeting efficiency of 1% or more, wherein the gene targeting efficiency means a ratio of homologous recombination efficiency to random recombination efficiency, the homologous recombination efficiency means a number of plants undergoing homologous recombination divided by a number of plants subjected to transformation, and the random recombination efficiency means a number of plants undergoing random recombination divided by a number of plants subjected to transformation.

5. The Agrobacterium according to claim 1, wherein function of a gene associated with T-DNA random insertion in the Agrobacterium is lost or reduced.

6. The Agrobacterium according to claim 5, wherein the gene associated with T-DNA random insertion is at least one selected from the group consisting of an ATU3081 gene, an ATU4309 gene, an ATU6150 (virII1) gene, an ATU6154 (virF) gene, an ATU6156 (virK) gene, an ATU6183 (virD3) gene, an ATU6184 (virD4) gene, an ATU6185 (virD5) gene, an ATU6188 (virE0) gene, an ATU6189 (virE1) gene, an ATU6191 (virE3) gene, an ATU6180 (virC1) gene, and a homologous gene thereof.

7. The Agrobacterium according to claim 1, which is Agrobacterium rhizogenes or Agrobacterium tumefaciens.

8. The Agrobacterium according to claim 1, which has a targeting vector comprising a foreign gene.

9. A method for producing the Agrobacterium according to claim 1, comprising:
disrupting a gene associated with T-DNA random insertion in a chromosome of an Agrobacterium by a homologous recombination method.

10. A plant cell, obtained by infecting a plant cell with the Agrobacterium according to claim 8.

11. A method for producing a transformed plant, comprising:
infecting a plant cell with the Agrobacterium according to claim 8.

12. The Agrobacterium according to claim 1, which does not have a T-DNA chromosomal insertion ability.

13. The Agrobacterium according to claim 5, wherein function of a gene associated with T-DNA random insertion in the Agrobacterium is lost, and the gene associated with T-DNA random insertion is at least one selected from the group consisting of an ATU3081 gene, an ATU4309 gene, an ATU6150 (virH1) gene, an ATU6154 (virF) gene, an ATU6156 (virK) gene, an ATU6183 (virD3) gene, an ATU6184 (virD4) gene, an ATU6185 (virD5) gene, an ATU6188 (virE0) gene, an ATU6189 (virE1) gene, an ATU6191 (virE3) gene, an ATU6180 (virC1) gene, and a homologous gene thereof.

14. The Agrobacterium according to claim 1, which has a reduced T-DNA chromosomal insertion ability compared to a T-DNA chromosomal insertion ability of the wild-type Agrobacterium.

15. The method according to claim 11, wherein the plant cell is a cell of a poaceous plant, a leguminous plant, or a malvaceous plant.


SEQUENCE LISTING (83 sequences)

SEQ ID NO: 1 - DNA, 1377 bp, Agrobacterium A208
ttgcggtcagaagtaaccgaacatgcggtgccgattccggttggcccgctattgcactccagcgccgatcttatggtctcgctcattgccttcaacaggcaggtgaaagccgaacgtgaaaaaccggcggtgaccgccgtctctgttgagaaggcgatgactgagacggacaacaggatggataaggctggaatggcggccgcgccgaccatgccgccacccatgtcaaaagacaaaccggccgacgtggccaataagagaccctatcttccccaacagatattgctggcggaggtcgatgcgcggtttaatggcaccgtccgccagcctctgatcgggtttggcgaacgtcttgcgatgttctgggcaaaccacttctccgtcgccatcggtaagggtggcgatgtgcacatcctcgccggcgccttcgagcgcgaagcgatccgcccgcatatattcggcaatttcgaggacatgctgcttgccgtggaaacccatcctgccatgttgttctttctcgacaaccagcaatcgatcggtcccaactcaccggccaacaaaaacggcaagcgcggcctgaacgaaaatctggcccgcgagatcatggaacttcacacgctgggcgtggatggcggttacaatcaggctgacgtgacggagcttgcgcgcatcatcaccggctggaccgtttatcgtgacgagaacaggccggggccggtcgggcgcttcagcttcaatgccaacagccacgagccgggtgatcacgcggtgatgggcatcacctatgccgatgccgggatggagcaggggcggcgggcgctgcgcgatctcgcccatcacccatccaccgcccgccaccttgccttcaaactggcaaggcacttcgtcagcgaccagccgcctgtggcgctggtgaccaagatcgccgctgcctacaccaaatcccacggcgatctcgccgccacctatcttgccatgctggatgcggaagaagcctgggatcccactctcaagaagttgcggccaccgctcgatttcgtcaccgccgtgctgcgcagcggcgatgtcaagccgaaacctgaacagatcgtttcggtgctgaaggcgctcgggcagcctttctgggacccctccggaccgaacggtttttccgatgtgagcgatagttggggttcctccgaggggctggcaacgcggatcgatgccgcgagcctttttgcccatcaggttgccggcggtatcgacccccgtgactttgttgccgatcggttcggcccgcttctcacgcgtgagaccgcccaggccgttcagcgcgcggaaaccaagccgcaagggctgaccatcgctttcctttcccccgaatttcagaggcgctaa

SEQ ID NO: 2 - DNA, 1032 bp, Agrobacterium A208
atgacctctcctcgcgccaagaaacgcctcagcgtcgccttcctgcttgccgatcgcttcacgctgtcggccttcgcgaattttgtcgatgtgctgcgccttgccgcagacgaggccgaccgctcccggccgatcctgtgtgaatggtcggtgctctcggcaacgctcgccaacgtgcagtcgagctgcggcgtgaaggtgcaaccggatacgcggctgatcgatgccggacactacgattatatcgtcgttgtcggcgggctgatcagcgaccacagcgccctgccaccggaagcgctacgcttcctgaaagatcgggcggcggcgggcattccgattgtcggactctgcaccggcgtcttcatcctgcacgaagcgggccttctcgatggctatcgctgctgcgtgagctggtttcaccgccaggattttctggatcgtttcgacacggtgcaaatggtctccgaccagatcttcgtcatcgatcgtgaccggctgacctgttccggcggccatggcgccgcgcatctggcggcctttctcgtcgagcgccatgtcggccagtccgccgcgatcaaaagcctcaacatcatgatgatcgacagcgcgctgagcggcgaaaaaccccaacccggccagcagagcgcgcgcaaggccaatgaccccctggtcaagaaggcgatcctgcggatgcagcagaatatcgaggttccgagaacggtgctggaactggcccacgacctcggccttggccgccgtagcctcgaacggcgtttcctcaatgacctgaaggcaacaccgtcaaaggtctatctcgagctccggctggatcgcgccctgtcacttctgcgcacgacgcatgacccgatcacccagatagcacttgccaccggcttctgcgacgcgccgcatctttcccgcacgctcaggaccgagcgcggcttcacccccagcgaatatcgcaagacccaggcgggccacacggcaaccgacgaggaattcgccgtcggcgtgctgcttccccaatcctga

SEQ ID NO: 3 - DNA, 1260 bp, Agrobacterium A208
atgatcacgagcagcatttctgggaccgaccagcagtttcaaaacgccacgcagccaaaagagctcgatcctgatgctgttcctgtaagcaggcttgattctgaaggccatgaaatttttgctgaatggcgacctaagcgcccgttccttcggagggaggacggcgtctttttggttctccgcgcggatgatatctttctgctgggcaccgatcctcgtacgaggcaaatagaaacagaactcatgttaaatcgaggcgtaacccgcggcgccgttttcgacttaatccgctacagcatgctcttttcaaacggcgaggtccatgtgaaacggcgctcagcctttgcgaagacctttgcattccggatgatcgacgcgctacgcccggagatcacgaagttaacggagcatttgtgggacgatgtaccgagagttgacgattttgactttgctgaaatgtacgcgtcgaaattgcctgcgctaacgattgccagtgtacttggtttgccatttggggatgcacctttcttcacacgacttgtttacaacgtttcgcgctgcttaagtccttcgtggggagaagacgattttccggagattgaggcttctgccgtcgagctccaggattatgtccgggcagtggtagcggatcgcagccgacgtataagcgatgacttcctctcttgctacttgaaagcggtgcgggaagaaggaacgctttcacccatcgaggagatcatgcaattggtgtttctcatactcgccggaagcgatgcaactcgtaacgcgatggtgatgctgccgactcttttgctgcaaaatcctgttgtctggtcctctctatgccacgatcaatccggcgtcgcggccgcagtggaggaagggcttcgcttcgagccatcagttgggtctttcccgcggttggcgctcgaagatattgatctggatgggtacgtattgccaaagggaagtttcctagccctaagcatcatgtccggcctgcgggacgagagacactacgagcatcctcagctttttgacataaaacgcaaacaaatgcgcaggcacctcggatttggcgcgggtgtgcatcggtgcctcggcgaagctctggcgcggattgaattgcaagaaggacttagaacacttctgcgccgcgcgccgagccttagggtgacaggcgactggccacggatgataggtcacgggggtgcacggcgcgccacggggatgacagtcaatctgggcgtcgatcggtga

SEQ ID NO: 4 - DNA, 939 bp, Agrobacterium A208
atggagcccagccaacgaagcttaagtgatgaatccgaagcgtcccgtacggaagtccattctacatcgacaaatagggatatgcctcccgagcttttggctaaggtggcaacttatattcctacccaggacccggtagaaacggcacgaaatcttggaagcctggaacgtacagggcgtgcaggtcgtgaagccgttacaagtgatcccgtcggaaaatatcacgcgcgaatgaaacgaattggcgcctccgccaaaaccgtgttcgatacggtcattcccggaaatcaattacctgagtgggagacgaaccgaccctctccgacggccagaacgcgagccgttggcccaattcttaagttccagtctgaagcgggaaaatcgagatttgtcaccaatatactcaatttgcctgaatctgctcagtgcgacgcaattcttagtgtcataaaacacttgaatgatcttggtgaagcaaacaagacgcgacttatcgaacgctctatcgaaattttgcggctagaaccccctttgacctggaatatgggccagaaatgccctgcggtcgacgtgctggtccaaggcgaaaagtacctaaatgcggatcaactggctcgcatacaacaagagaggagcagccgtccgcggctgtctcccctgtttggccgagccatagccgatttcgaggttcaaaaaaccatggacgcaaatcctagacgatatagggacgcggcggggcctgaggtcagagctgtagacacagtaattgagacaattgagagatatagtgcgtcgttagcaagaggcggtccaaatgcagtgggactcctaacgacgaacgaaagctttgagaaaaatatcaatgaggtctacaaccgtacgcgagccgagctagacgcttcttcacgtgacagaagtcgctctggactatcgcgataa

SEQ ID NO: 5 - DNA, 2502 bp, Agrobacterium A208
atgaatggaaggtattcaccgtctcggcaagatttcaagacaggcgccaagccttggtctatcctggccttggtagttgctgcaatgatttttgccttgatggcgattacgtcttggcaggacaatgaaaccaatcgggcgatcctgacccaattgcgagctattaacatcgacagtgcttcgctgcagcgggatgtactccgcgcggaagcgggtgtggtggcgaactaccggcccattatctccaggttgggagctttgcggaagaacctggaaaatttgaagcgactatttaaacaatctcatcttgtgatcggcaatgatttctctcaactgctcgacaagctaaaggtgtctgtggatacgaccgacgcggccgttgcagccttcggagcgcaaaacgtgctcctgcaagattcgcttgccagcttcactcgcgcgcttagtattcttcccaaaatgtcgtcgacggatcagacggtcgaaaattcgaacgaattgggcagcctgatgctgcggtttgtgcgtcagccaagcccagcactctcgttggagatcagtcacgaactcgacatgctccaaaaagctagcggtggggctgaagttcctatccgtatacttgcacgcgagggtcgcgtcatcttgtcgattttgccccgtgtgaacgatgccgtaaacatgattcagacctccgacaccgctgaaattgccgaaagattggagcgcaagtgtttggaggcctatagcttgcaaagcgtgagggagcagcgggcacggatcttcttgggttccgtttcggtgggcctttgcatctacatcatctcactggtctataggctgcgtcggaaaacggcttggttaacgcggcgtttggattacgaagaggtaatcaaagagattggggtttgtttcgaggggggaggggccacagcgtcgtccctcaattcgtccgcgcaagctgcgcttggaattattcaacgcttctttaatgcggaatcgtgtgcactagcattggtggaccatggtgacaggtgggctgtcgaaagtttcgctgcgaagctgcctgagcccgtctgggaggacctcgcgctacgcgagatggtttctcttgccagagcggatgagcgtgcgtcagtattccgcatcatgtcgacgcgaaaggtcagctgccttcctccggagactccgggcgtttctatgctgctggcacacaaatctacggatcaactgatagcgatttgttccctcggttaccagggctatcgtctgaaatcttgtccaggcgaagttcagcttcttgaactcgccaccgcctgcctctgccactatatcgatgtccggcgtaagcagaccgaatgcgatattctggagaggcgattagagcatgcggaacgtcttcaggcagttggtacacttgctggtggaatagcgcatgagttcaacaacattttgggagcaatcctcgggtacgctgaaatggcgcaaaacatgctgcgtcggtcatctgtcacccgaagacacattgaccaaattatttcgtcgggtgacagagccaggctcattatcgatcagatcttgactctaagtcgaaaactagaacgcgtgacaaagccgttcagtgtctccgaactcgtaatggaaattgctcccttattgcgtgttgcattgcagcgcaacatcgagctgaagttcaagtttgacgacaagaagagcgtagtcgaaggaagcccgctagaggttcagcagatgctgatgaatctttgcaagaacgcttcccaggcgtttaccgccgatggtcaaatcgacatcatcgttagccgaatttttgtatctcgacagaaagtactggcgcatggtgttatgccagctggcgactatgttcttctttctgtcagcgacgacggtgaaggcattgccgaaactgtgctcccccacatttttgagcctttctttacaacacgctcttgcagcggcggcacgggtctaggtcttgctgccgtgcacggtcatgtcagcgcgcttgcaggatatattgacgttacttcagccgtagggcgagggacgcgcttcgacatttatctacctccttcctcgaagaagcccgtcagcccggacgcgttttttgggccctgtaaaacaccgcgtggcaacggagaaattgtggcattgattgagccagatcctgtcttgcgggaggtgtacgaagacaagatcgccgctctgggctatgagccggtgggctttaagacatgtgcagacctttgcaattggatatcaaaaggcaagcaagccgatctggttctagttgaccaatcgtctcttcccgagaatcagagtgctactgctttgcacgcagccttcaagacggcgtccatcatcattggaggaagtgatcttaaaatgtcactttccagcgatgacatgacgtccgcgctttttctgcccaaacctatatcctccaggacgatggcctacgcgattcgtaccaagatcaaagcctag

SEQ ID NO: 6 - DNA, 2022 bp, Agrobacterium A208
atggcaaatgaagagttcaccagacactatgcgtggcccgttcctgtggcttcgaatgatgaagggcgtgggaccgcgcggatccccatccaggcacaatcaatcgttgctggagaagacggtcgggacacttcggtcccaacggctttgtcgcgaccgccaattgaagatatgccgcacggcgtccaagaaacatcggcgagtggcggacgactgggagcggcccgtctgcgggattccgtaatcccgccaggaatatccgaagcccgcacggacctatccgcaattttgcggaaaaaaagcggttctttccgcaccggtatgcagtatctgcgtgggcttgaacgggaaaattttgataaacaagacagggaagccagtgcgttgccagatttaagtgcaaggggcataaagcgaccgcgcgaaattgagtatcccggcaatgcaagcggattaaccataaagaggcaagacggcttaggcatagagatcaatactatctcggcatcttcgcccgtgaaccgggccgcgcattcgtcgaactggcaaggcgcgccggaaccgggcgtgtacaatgttcagccatcggcagatagagcacagaactctgcgcaggaaagttcgacgtttcccgatggcacttctgtttccgctctctattcaggcccactcgcagaatggttcgagagggatactggcagcgagacgaccaggaattcaggcaacaccatttcgtcaccgctccgagggctggaggagttcggcgattccgcggacagccggtatcttgggcgcgaagctcagagtctttcagttaccgtaacaacgcctaattcgaatgctgaggcaagttctcatagcgcacacactgaaactctcgacgatgtcagcagtgaccgctcgagcgaacaggggaggggtccccttggcgccgcaatccttggatcccatcatgacctttcaccgcgtgcgcagaagttatcacaaacaaaccgcgattcccctgaactgaccgacgccgacctggcaaaagtcgatgcggtgttcgaatctctctccaaggggcccccggcgggggagagcgccgcaccggactttcgtgagcgcggaccaggcagcgctttccagaaagaaggcgtgtccgacagggcgaacggggtgcccactaactgggaggtaccttttggtcgcggtggcgggcattcaccgcaagcgctgaggtcctcaggagtggagctcgatgacttccctgatttcaccgaggccgaactggcaaaaatcgacgcactagtcgaatctcactccaacagatctttatcagtgcgaaatatagtaccagatttgcgcggagcgggagcagacaacgtcttccggaaagaaggcgttgtcgaacgcgcggagaagatgccgatcgatagtgtgagtcttaccagactgaatggcgagcgttcacgttcaccgaagacatcgcaagcaagccttgaggatttccccgatctaaccgatgccgatctggcccatatcgaagagtcggagcggatcgcaagaactgctgttgaaaaggggaagcaaaaaatctcgaccgaagccgatacgcggtttgatttgggcaactcttctgcaccccgggttagccctcgatcggttaccccgttggttcccaatgccaatcagccgattacatcgtggttctacgaggcacaaaagacttgtgacaaattggtagagaacacttacgtgaagccggccgttgacagttctagggcgcgaaatgacgtcgaaaacaccgctgccaggcttggtgaccctgctcctgctttggggcatgacaatcttggtagaactcgcgcgttaacgcccgttcgcgacgtgatgtctagacccagcgcggaccgacaactggctagtcatgcggcagaacattcggcaatagacgacatttggaaacgggatgaccgtgatcgcaggacacacccttatagaggattggattcgcgttcacgtgaaggttgcgggcggtaa

SEQ ID NO: 7 - DNA, 2007 bp, Agrobacterium A208
atgaattcgagcaagactacgccccagcgtttagctgtcagcatcgtatgttcgctggcagccggtttttgcgcggcaagtctctatgtaacatttcgccatggtttcaatggcgaagcgatgatgacgtttagcgtcttcgccttttggtacgagaccccgctctatatgggtcatgcgacccccgtcttctattgcggtttagccattgtcgtctcgacgtctattgttgtgctgttaagccaacttatcatatcgtttcgcaatcacgagcatcatggcacggctcgttgggcgggatttggcgaaatgcggcacgccggttacctgcagcgctataatcgtatcaaggggccaatctttggcaagacgtgtggccctcgttggttcggcagctatctgaccaatggcgaacagcctcacagtcttgttgtcgcgccaacgcgcgccggcaaaggcgttggcgtcgttattccgacgctcctaaccttcaagggttcggtgatagccctcgacgtcaaaggcgagctttttgagctgacttccagagcacgcaaagcgggcggcgacgccgttttcaagttctcgcctctggatccagagcgacggactcattgttacaatcccgtcctcgatattgcagctttaccgcccgagcggcagtttaccgagacgcgccgcctcgccgcaaacctcatcacggccaaaggcaagggagcggaaggctttatcgacggcgcgcgggaccttttcgttgcgggcatccttacctgtattgagcgcggtacgccaacaattggtgcggtctacgacttatttgctcaacctggagagaagtacaaactttttgcgcacctcgcggaagaaagccgaaataaagaggctcagcgcattttcgacaatatggcgggtaacgacacgaaaattttgacttcttatacatcagtgctcggcgacggcggacttaacctgtgggctgatccattggttaaagcagcgacaagtcgatcagatttttccgtctatgatctgcgtcggaagaggacctgcgtctatctttgcgtcagtcccaacgacctcgaggtcgtggcgccattgatgcgcctcctttttcagcaggtcgtgtcaattctgcagcgatcactgccaggtaaagatgagcggcatgaagttttgtttctccttgacgaatttaaacacctggggaagcttgaggcaatcgagaccgcgattacaaccatcgccggttacaaaggccgcttcatgtttattattcagagtctctcggccttgacgggcatctacgatgacgcgggcaaacaaaactttctcagtaacactggcgtgcaagtatttatggccacggccgacgacgaaacaccgacctatatctcaaaagctatcggcgactatacgttcaaggcgcgttcgacctcatacagtcaagcccgaatgttcgatcataacatccagatttccgatcagggggcaccgcttttgcgccccgaacaagtgcgcttgcttgacgataacaatgaaatcgtccttattaaagggcatccgcctctcaaactacgaaaggtgcgatattattccgatcgtatgctgaggcgccttttcgaatgccaaattggcgccctccctgagcccgcatctttgatgctttcggaaggtgtccatcgggatgggcaagacctcagtcaacaagccgccgtcacggaggcgcagggcttaggtgacatcgattcgatacctaataatatggaagccgctacaccgcaaaacagtgaaatggatgacgagcaggacagcctcccaactggaattgacgtcccccagggccttattgaaagcgatgaggtgaaagaagacgccggtggcgtggtgccagatttcggtgtttcagctgaaatggctccagctatgattgcacaacagcagctgctggagcagatcattgcgcttcagcaacgatatggacccgcgtcctcgcactcggtgaaatga

SEQ ID NO: 8 - DNA, 2523 bp, Agrobacterium A208
atgagaccttcaggaaacccgaacgtcgatcttagcggttcgactgcatcgcttgccgaagttcccgccggagctacccctgtccttaatctaatcgagcccaggaaccgtccggctgacgactcgcttgagggccaaaccgatcgcggcgagcatccatctgcatcatttgactatgatggcatgaagcttggcgccgcggagcgtgaagcatacgagaactggtgtccatcgaaccggcctacatggaaagatctggtactcagggcgcgccttgatgcaatcgacagttccgcttggctccccgatttgggcgaggagtcgcctttgatcttcagatatgaagggattccgctgggtgagggggaacggcaagcctacaaagaatggcaagaggaggctcagcccacatgggaagacctcgttgtcaacgcacgaatggcggtacctgatccttgtgctgacgttgcagacgagcacaatcccctcaaagaaggcgaggagtttcggtctgaagcgtcgaaacgcaagcggaaaaaaccgatcgaccaggacgagaattctcctacatcgttttactatgacgggatgaggctcggagaacccgagcgcgaggcatatgataactggggcaacgcggagccgcccacgtggaaagacctggtacttaaggcgcgccttgatgcaattgacagctccgcctggctctttgcttcagaagggtcttcctcgacttttgagtatgagggaattccactgggtgagggggaacggcaagcctacaaagaatggcaagaggacgctcagcccacgtgggaggacctcgtcattaatgcacgcatggcagaactcgaccatccttcttggattacagacgagcacaattcccttgaagaaaacttagagtttcggcccgatgcaagacaggccagcctgaaggactcgaccgaccagcggaagagttcttccgcgtcatttatctatgatggaatgaagctcggggaacccgagcatgctgcatacgagaactggagcaaaccggaacgaccgtcatgggaagccctcatcctagatgcgcgccaggcttccatagcaagctcttcggtttcgaattcgttacttgcaaagacatcctcgccagtctttctatacgagggaatgtcgctaggggatgcggaacgtcaatcgtatggaaggtggaggcagcctgcccaaccgcgatggcaaaatcttgtggtgaacgcgcgccttgcggatcttgatccgttggcctggattcccgatgaacatgatccgtttgcggaagccgaggcgcttagccccacttcgcaatcgagtggcgccaacaagtccaaccgcgctttgggtcaatcagattcaggccggcccgccttcgcatatctggcagcacaagaggcgagtcacctgcaatcaccggcatgctcacaattggaaacgaggcgtgcattaaatttcgggtcgcctggaccggatgcaaatccaacggaaagcatcgccaaatgtaatcgtttggatggcgtcagtaaaattaagcgattaggcaccaaaggccgccgggcagtaaacgcgactattcacggcggcaagttcggtgcgcaaggactgttgtccgaagattgcggacaagcggctgagccctctccatcggaacaaactactcggccgcgaaccgataatattggtacctatgcaagccggaaaaacgaacgagctcggctggctacagagaccgggaaatacgaatcggagcacattttcggatttaaggtcgtccacgatactttgcgggcgaccaaagaaggccgtcgtctcgaaaggccaatgccggcatacctcgaatacaaggagcttcatcggcaacacgtagggacaggaagaggacggactggactggtcgggcgcggatggccagatgatgcgagctatcgcgcagatcaaagggcaaccctgtcggaccctgttgcgtccgcggaaggtgcaacagcttcaaatgggtaccagttgaaccagctgggctacgcgcaccagctcgccacccatggtctccaaagtgaaacgcccgatggggttaccatgccaattcaagttgcgacaattagctacaactatacagtgagtcgtgatccggtcctttcaccacccagcaaagaacaagcgcccccattgctacaccttgggcctcgtgggcaaacggaagctgtgcttgcccgcgagaccgcattgaccggaaaatggccaactctcgagcgtgagtgtcaagtgtatcagcggtttttggccctgtacgacgtaaagaaggatctggacgccaaacaacttggtgtacggcagaaaaagcaggcactcgttgcggcgttgaaccggaccgccggcttaattggcgcgtcacctttggaagcccaatcgtcgaccgctgaggttgaatatactaccgacgagcccgatgaacgacgggtttatgatccgcgcgatcgaggcagagacaaagcgttcaatcgctga

SEQ ID NO: 9 - DNA, 252 bp, Agrobacterium A208
atgcacggtgatgatgttgatcggacggctttcgtcccgcgcaggctgccggctgaagccgcttacgtcgatagtcaagttgacctggcggcaaaccgacaacctcactgtgtgacttgttttgagcgacttttggttcaagagaaagatgaaaaccgtcgacgattgtgggcttccggcaaagcatgcgccacgtctgaagagagttgtgcaggaggccctacactcgcaccgggccattcggggccatga

SEQ ID NO: 10 - DNA, 192 bp, Agrobacterium A208
atggtgatcatcaagctaaatgcgaataaaaacatgcctgtcttggcggttgagaagccgcaagaaattcacaaagaggagttgagcgaccatcaccagtcaaatggctttacgagtttggatctcgaaatgattgaactggagaattttgtccttcactgcccgctccccgaagaaaacctagccggctaa

SEQ ID NO: 11 - DNA, 1671 bp, Agrobacterium A208
atggatccgaaggccgaaggcaatggtgaaaatatcactgagaccgcagcaggcaatgtcgaaacttctgatttcgtgaatttgaagcgccagaagagggagggcgtaaattccaccgggatgtccgaaattgatatgacgggtagccaagaaactcccgaacacaacatgcacggaagcccgactcacacggatgatctcggcccgcggttggatgcggacatgctcgattctcagtcaagtcatgtttctagcagcgctcaaggcaatcggtctgaggttgaaaatgagctatccaacttattcgcgaagatggctttaccaggccatgatcggcgtaccgacgagtatattcttgtgcggcaaaccggacaagacaagttcgcaggtactactaaatgtaacctcgatcatctgcccaccaaggcggaattcaatgcgagctgccggctctatagggatggagtcggcaactactatcccccgcccctcgcattcgagaggatcgatatcccggagcaattggctgcacaattgcataacctggagccaagagaacagagtaaacagtgttttcagtacaagttggaagtctggaatcgcgctcacgcagagatgggcatcactggcaccgacatcttctatcaaacagacaagaatattaagctcgaccgaaattataaattgaggcctgaggatagatatatacaaacagagaaatacgggcgcagagaaattcaaaaacgctatgagcaccagtttcaagctggttcactgctgccggatatcttaatcaagaccccgcaaaatgatatacatttctcgtacaggtttgcgggcgacgcttacgctaacaagcgatttgaggaattcgaacgcgcaatcaaaactaaatacggtagcgataccgagatcaagctcaaatccaaatctgggattatgcatgactccaaatatttggaatcatgggagcggggcagtgcggatatccgtttcgcagagttcgccggcgagaatcgagctcacaacaagcagtttccggctgcgactgtgaatatgggaaggcagccagatggccagggagggatgactcgcgatcgccatgtaagcgttgactacctattgcaaaacctacccaactccccttggacgcaagccttgaaagagggaaagttgtgggatcgagttcaggtccttgctcgcgacggaaaccgttacatgtcaccttcaagactggaatattccgaccccgaacactttacccaactgatggatcaagttggtctgcccgtgtcgatgggtcggcaaagtcatgcgaatagtgtcaagtttgagcagtttgacagacaggcagcggttattgttgcggatggcccgaacttacgtgaggttccagatttgtccccggaaaagttgcaacaactgtctcaaaaagatgtcctgatagcggatcgcaatgaaaaggggcaaagaaccggcacttacactaatgttgtggaatatgagcgcctgatgatgaaattaccgagcgacgcagcgcagcttctcgctgaaccgtccgatagatattcacgtgcttttgtccggccggagccagcattgccccccatcagtgacagccggcggacttatgaaagccgaccgcgcggcccaaccgtaaacagtctgtag

SEQ ID NO: 12 - DNA, 2055 bp, Agrobacterium A208
atggtcgacacgacgaagaagagtgtcgcgaagtcgcttacggctgacatgcgccgttctgctaagcggctttctaagcaaatgcgtaaagcctcgcttactgaagaggaggcaacaaggaatctagcccggctcgaaacgccggaccagaagcgaaaatatgtcgccgatatgcagataatcgacaagctggaagacggctttcgaggcgaaataagctataaaatgctgggaaataaacagcttcgggtcgacagcccaaaagaattaacgcgcgagcatggtataataagaaaaacaagaaaggttctgaagcgtaacgcagagactggcaatgtttacttgggtctccacgaaaagaagacctggaggagcgttagcagccatctttatgccgaggacggtacacttcgcgcgaagcatgtgaaatacaaagacggacgctttgaagaaaaatgggaacgagacgaaaatggcctgctgttccgcacgcagtttgtcaaccgaaatcggctatttcaacctatttccgagaaggtcagcacaccctaccggagcggaccggaaaaccggctctttcgtgaactaacccgtcgaaaaggttccaagcaggaaacttttgagcgggacgaaaaaggcaacctcgagctcatcggcagcaaacgtctcggcttttccaagaattcgacgaaagccgtggatcgtcagacctctcagacgacgattcgaaaacttggtggcgcattcagcaaatcttatagatccctgctagacacagagggcaacgaacttggccgagatatatcgagtcatcgacggctcttcaacaagcgatcggctgtctacgatgaggctagcgggcaattgacgagcaccaagcatacgttcggtaagatctacaagagtgaaacagcgtatttgaacgctgatatcaaaaaagtctcaaaaaagatactcggcgtgacggttcgtcggaaactgacgacgctaagtgaacaagaacacgacgctcagaagctgcgaaattcggaatccattgagcataggaaggcttggcaagagcgcgcagttatccctagatcgcctccacgggagacggcaaatgtggattcggcgccgcagtcgcatctggtcaactctcttagagacggccgtcgtgatgaggctgagcctagggttgtgcctgcaaaagaccgacgattagaaggcgttatacaaaattcacccttgtttcggcaaccgaactccgagcgacgggtcggttcccaaacactacctccatcgtcaaatgtgcgtgcctcagatccagtttttccaagggaaggcgctaagtcggaaaaagggccagtggcggtcaatctccagcgtgataatgaaagggaaaagcaagaggaaaaaaacctcagtgagaggcggggaaatctcttccggtcaagccggtcaataacagagacgtcgtttcccggatcgccggaaagaataacgatgttaggttcgcggcgggcctcgataaacccatccgccgatccgcaatcgccggtcggcagaacggattcccaagcgccgccgatgtccttatttccgatcaataatcatcagtcggatccaaacgagcaagagctcttgagtttcctgcatagcgtgccagtcccgccgcctttggaagtacatggcgcaccaggcgggcgtgttgaggacagcctccgtggagcttccggagacagcctggtgcgaacgagttcagtgtcggagagtgtgttcgatgaacattcgcaagggcctttggaacgcgatcactcgaccaatgcgacaagtgagcgctttgatccgcaggctttgtttggcgaaccgggcttgtcgcgcgtctcagaaacacgaccagaactctcgatgcaaggcgaccatttgacaaattcggaacagcaagcgttactgaatgaactgcttagtgtgccattaccgggtcctttgccgaaggcggatcatgagcgaccgagggttttggaaagctcacggagccgagagcgctccatgtcaggagggctttcactttag

SEQ ID NO: 13 - DNA, 435 bp, Agrobacterium A208
atgaaacttatgctacccaccctgtttgggatacttttggctacggctgcaaacgctgccaatgatagtcatgtactagcaacctttcccgagattgcgcttgcactgactaccggcaagccagtggacgttatggtcgatttaagtttgtgtactccggggaccgacgatacaccgccgacgaagacgcgcggaggcattcgcatagatgggtatagaatcacttctgacggcaccctcgcttttgcggatcagcactttacgattgatcgtgacggtaagcctattcttcagtttcttcgctaccaaatccggttggacggtgacgccgaactgactatggtcgtattcaacatgccaagctacgagagaaaaggtaccagcttggcctacaagtgcgcgatcagtcacgggttgagtttcttcgctccatag

SEQ ID NO: 14 - DNA, 696 bp, Agrobacterium A208
atgaaacttctgacattttgctccttcaagggaggagccggcaaaaccacggcactcatgggtctttgcgccgcctttgcaagtgacggcaaacgattggctctcttcgacgctgatgaaaaccgaccactaacgcgatggaaagaaaacgcccttcgcagcaatacctggggctccttctgcgaagtctacgccgccgaggaaatggcactccttgaggcggcctatgaggacgccgaactccagggatttgattatgcgctggccgatacgcatggtggttcgagcgaactcaacaacacgatcattgccagctcaaacctgcttctgatcccgaccatgttaactccgctcgatatcgatgaagcattgtcgacctaccgctatgtcattgaactgctgctgagcgagaacttggcaattccgacagccgtattgcgccaacgcgtgccggttggtcgattgaccacatcgcagcgcgcgatgtcggacatgctcgcaagccttccagttgtacagtctcccatgcacgagagagacgcatttgccgcgatgaaggaacgtggcatgttgcatctcacattgctgaatatgagaaccgatccgacaatgcgcctcctcgaacggaatctcagaatcgccatggaggaactcgtcactatctccaaattggttagcgaagccttggaggggtga

SEQ ID NO: 15 - DNA, 1269 bp, Agrobacterium  EHA101
atgattgcgaacagctcttccgatgtctctatggccgaccagaagtttctaaatgtcgcgaagtcaaatgaaatcgatcccgacgccgttcctataagcagacttgattctgaaggtcacagtatttttgcagaatggcgaccgaagcgcccgtttcttcggagagaagatggcgtctttctcgttctccgcgcagaccatatctttctgctgggcaccgatccacgcacccggcaaatagaaactgagctcatgctgaatcgaggcgtcaaagctggtgccgtttttgactttataggtcacagcatgttgttttcgaacggtgaaacgcatgggaagcggcgctcaggtctttcaaaggcgttctcattccgcatggttgaagcgttacgcccggagattgcgaagataacggagcgtttgtgggacgaactacaaaaagttgacgatttcaattttactgaaatgtacgcgtcgcaattgcctgcgctgacgatcgcaagtgtccttggcttgccgtctgaggacacgccgtttttcacacgacttgtttataaggtttcccgctgcttgagcccgtcgtggcgagatgaggaattcgaggagattgaagcttccgctatcgagcttcaggattacgttcggggcgtgatcgcggatagtggccgtcggatgagggatgattttctctcgcgctacttgaaggcggtacgggaagccggaacgctttcgcccattgaggagatcatgcaactcatgcttatcatactcgctgggagcgatacaacgcgcactgcaatggttatggtgacggctcttgtgctccaaaatcccgcgctctggtcttctttaactggcaatcaatcctatgtcgcagccgccgtggaggaagggctccgattcgagccgccagttggctcttttccgcggttggccctcgaggatatcgatctggatggatacgtgttgccaaagggaagcctcctcgcgctcagtgtcatgtctggcctgcgagacgaaaaacactacgagaaccctcatctttttgatgttgggcgtcaacaaatgcgttggcacctcgggtttggcgcgggagttcatcgttgcctcggcgagacgttggctcggattgaattgcaagaaggacttggaacacttttgcgccgcgcgccgaatcttacggtggtaggtgactggccacggatgatgggtcacggaggcatccggcgcgccaccggcatgacggtcaaattgagcgtcgaccggtga

SEQ ID NO: 16 - DNA, 942 bp, Agrobacterium  EHA101
atgcagaatattgtatccaattcacagacacagcggcaacccatagaaaaagaggtgtcacccttccatcgcgtgatgagcgacgatctgatctcccggatcgtggatggcatggtcactaacgatcctgttgagactgcaaggaatatctcgaaccttaagctctctaataaatctgtaaaggaaagtgttgagcgtagtgctggaggaagatttcacggacaaatcaatagacttggcgcagcaagcaaggccttatatgaccacgcggttcccacagaaggattctctgagcatcctgagtttcgtccagccttgccaagggactacgccctggccggggagagaattgatgcaatcgggggaactttaaagctgcaaacaccgcaacgaaaatcagcgctcgtagatcatatattgaatatcgcagaaccgtacgatcaaggtcacgcgcttgagtatgttgcgccccatgtcggtgaatttagctcggaagatcgtcgacgtcttgtcgagaaaatgcttgagcacttttccaccccctttaccgatgagttgccaagcgatgaaaacttcagtggaacttacgccttgttcaaggcattgccccacatggaagatagcctgaaggcgaggatgcttgataccgtggtcaataatcctgatatggcagcactcattcaatcgcagaaggaacgctatgattggttgagggcgcaagatgagtacgagaaggccacttggcgctccacgcaaaagccaactgtcgatgagcggctggcagacatagaaagttcagttagaagcaatctcagcctcggtcaaggcagtgaccttgagaagatgcgctctgcaaggcccctgcaacagactataagtgacaccctcaatctcgcgcgtgaagaactcaaaagccgcgcccccgacgtccgagggaggtaa

SEQ ID NO: 17 - DNA, 2498 bp, Agrobacterium  EHA101
taagtgcgatgaacggaaggtattcaccgacgcggcaggattttaagacaggcgcgaagccttggtctatattagcccttatcgttgccgcaatgattttcgcgttcatggcggttgcgtcctggcaggacaatgggactacccaggcaatccttagccaaatacgatcgattaacgctgacagcgcctcactgcagcgcgatgtactccgcgctcacacgggcaccgtggcgaactaccgccccattatctccaggctgggagctctgcggaagaatctggaagatttgaagcaattatttaggcaatctcatattgtaagtgagagcaatgctgctcaactgctacgccggctagaagtgtctctaaattcggctgacgcggcggtcgccgcctttggtgcgcaaaatgtccgcctgcaagattcgctggccagtttcactcgtgctttaagcaatcttccaggaaaggcctcaagcgatcagactttagaaaaaccaacagaattggctagcatgatgctccaatttcttcggcaaccaagcccggctatttcattcgagatcagccttgaactagagaggctccaaaaacaacgcggtcttgatgaagctcccgtgcgcatactttcgcgtgaaggtcccattatcttatcgcttttgccacaggtgaacgatctggtgaacatgattcagacgtctgacaccgcagaaattgcggaaatgttgcagcgtgagtgtttggaggtctatagcttgaaaaatgtggaggagcggagcgcacgtatctttcttgggtccgcttcagtgggtctttgcctgtacatcatcaccttagtctataggctacgcaaaaaaaccgattggttagcgcggcgtttagattacgaagagctaatcaaagagatcggagtatgttttgaaggtgaggcggccactacgtcgtccgcgcaagctgcacttggtattattcagcgcttctttgatgccgatacgtgcgcgttagctctagtggaccatgaccgtaggtgggctgtcgaaacattcggtgcgaagcacccaaaacccgtgtgggacgacagggtgctacgcgaaatagtctctcgtaccaaagcgaacgaacgggcgacggtattccgcatcgtatcgacgcaaaaaatcgtacatttgcctcccgaaattccaggtctttcgatactactggctcacaaatccacagataaactaattgcggtttgttcactgggttaccaaagctatcgccctcgaccttgccaaggcgaaattcagcttcttgaactcgccaccgcctgcctctgtcactatatcgatgttcggcgtaagcagaccgaatgcgacgttttggccagacgattggagcatgcgcaacgccttgaggcagttggtacacttgccggcggaatagcacatgaatttaataacattttggggtcaatcctcgggcacgcagaattggcccaaaactcggtgtctcgaacatctgtcacccgaagatacattgactatatcatttcgtcaggcgacagagccatgctcattatcgatcagatcttgacgctgagccgaaaacaagagcgcgtgatcaagccatttagtgtctcagagcttgtgaccgaaatcgctcccttgctacgtatggctcttcccccaaccatcgagcttagtttcagatttgatcaaatgcagagcgtgatcgaaggaagcccgcttgaacttcaacaggtactaattaacctctgcaagaatgcttcccaagccatgactgcaaatggtcaaatcgacatcttcgtcggccaagcttatttaccagctaagaaaattctggcgcatggtgttatgccacctggcgactatgttctactatctgtcagcgacaatggtggaggcatttcagaggctgtgctaccctacatttttgaacccttctttacgacacgtgctcgcaacggtggaacgggtctcggcctcgcttctgtgcatggtcatatcagcgcgtttgcaggttacatcgacgttagttcaactgttgggcatgggacgcgctttgacatttatctccctccgtcttctaaggagcccgtcaatcccgacagctttttcggccgcaataaggcaccgcgtggaaacggggagattgtggcacttgttgagcccgatgacctcctgcgggaggcgtatgaagacaagatcgccgctctgggatatgagccggtcggttttcgtacctttagtgaaattcgcgattggatttcaaaaggcaatgaagccgatctggtcatggtcgaccaagcgtctcttcctgaagatcaaagtcctaattccgtggatttagtgctcaagaccgcctccatcatcattggcggaaatgatctcaaaatgcccctttcaagggagaatgcgaccagggacctttatctgccgaagccgatatcgtccagaactatggcgcatgcaatcctaaccaaaatcaagacgtag

SEQ ID NO: 18 - DNA, 639 bp, Agrobacterium  EHA101
atggcaaatggtcagttcacgatacgctctgctcgcccggcctccgtcggactgacaggcgaacggcgtggagccgcatccgcctctagctctgcactgtccaatgttcaaagagatgttagggataggctgattccaactagctcaccaagattaccaaatgcagccatattgcgtgattcctcgggaagagcgtcgactggtctgcggtacatggcggctactcttcattggtctgcgatcgcgccattatcgctaataaacagcaacgacctggctccggccgcttatgactttgagacgcgaaataacgcaagaaatgtgactgccaaagtcggcagggcagtccctgttcccaagcaaggcgggctcggcaaaacgctcgcacccgtaccccttagtacacgtatatcaagggtcaattccgaccgaagactgcccgctgacgcagaagaccgccctgaaacgcgcgacccccagaaaggacgtggcagtcatggtgcgacgccaaccttacatgaaaagattggaaccgcgtttgctcgaagattgcgaaagcatacgtactatattgtttgcagttgctgccagacccggagcgcgttgacgatgggtgcaaagatttcggtgaagtcatga

SEQ ID NO: 19 - DNA, 1959 bp, Agrobacterium  EHA101
atgaactccagcaagacttcgccccagcgtatgaccctgagcatcgtatgttcgctggcagccggtttttgtgcggccagctgctatgtaacgttccgccggggcttcaacggcgaagcgatgatgacgttcgacgttttcgctttttggtatgagaccccgctttacttgggttatgccagcaccgtcttctggcgtggtttatctgttgtcatctttacctcgctgatcgttctttcaagtcagctcatcatatcgctgcgcaatcagaagcatcatgggacagctcgttgggcagaaattggcgaaatgcggcatgctggttatctgcagcgttacagtcgcatcaaggggccgatctttggaaagacatgtggtcccctttggttcggcagttatttgaccaatggcgaacagccacacagtcttgtcgtcgcgccaacgcgtgctggcaaaggcgtcggcatcgtcattccaacgctgttgaccttcaagggctcggtaatcgcccttgacgtcaagggagaattgtttgaactgacgtccagagcacgcaaagcgagcggcgacgcagttttcaagttctcccccctagatcctgagcggaagactcattgttacaatccggtcctggatattgccgcacttccgcccgaacgccagttcactgaaacacgccgtctagctgcgaaccttattacggctaagggaaagggagcagaaggctttattgacggcgcacgtgacctgttcgtcgcgggaatccttacctgcattgagcgtggcacaccaacgattggcgcggtatatgacctatttgcgcagcctggcgaaaagtataagctttttgcgcaactcgcggaggaaagcctaaacaaagaggctcagcgtatcttcgataatatggcgggcaacgacacgaaaattctgacatcgtacacctctgtgctgggcgacggtggactgaacctgtgggttgatccgcttatcaaagcagcgacaagccggtcagacttttccgtttacgatctccggaggaagaagacctgcatttatctttgtgtcagtcccaacgatctggaggtcttggcaccacttatgcgcctgatgtttcagcagctcgtgtcaatcttgcagagatcgctgccaggtgaagacgagtgccatgaagttttatttctcctcgacgaattcaaacacctgggcaagcttgaggccatagagaccgcgatcacaaccatcgccggttacagaggccgctttatgtttattattcaaagtctttcggccttgtcgggcacatacgatgacgcaggaaaacaaaactttctgagcaatactggcgtacaagtatttatggccacggctgatgacgaaactccaacctacatctcaaaagctatcggcgaatatacgtttaaagcgcgttcgacctcttacagtcaagccagaatgttcgaccacaacatccagatttctgatcaaggtgcagcccttttgcgccccgaacaagtgcgcctgctagacgatcagagtgaaatcgttctcatcaaagggcgacctccactcaaattacgaaaggtgcagtattattccgatcgtacgctgaaaggccttttcgaacgccagatgggctctctgcctgagcccgcacccttgatgctttccgactatagcaacgatcaagttcaataccacttggctccgatagcaaattttaatgaggatgctgcaccgcaaaacagaactgtggccgaggaccatggaagtgttaaagtcggtgctgatatccctgaacgcgtgatgggaataaatggtgacgaggaacaagccgatgcgggcgagataccgccggaatcggttgtgcctccagaattgacgctcgctctgaccgctcaacagcaattgttggaccagattattgcacttcagcaaagatcgaggtccgcaccggcatag

SEQ ID NO: 20 - DNA, 2511 bp, Agrobacterium  EHA101
atgacaggaaagtcgaaagttcacataagaggttcggctgacgcgcttcctgacgttcctggcggaagtactaccgccccttttttaaccgaaccttctcgggatcaggttgatgcctcgtttgaggtccaaaccgactacagccagtctacttccgtgtcgtttacctatgatggtgttggacttggtcctgccgagcgtgcggcttacgagaactggtgcgaaccgggccggcccacttggaaagatcttataatcaaggcacgtgtcgatccgattgacgatgtgacctggctccgagatttagaagaggacaccccctcaaccttcagatacgaagggatgcctctgggcatcggggaacgacaggcctacgaaaattggcaagaggacgctcagccgacatgggaagaccttgttgtcagcgcacgcttgacggaacttggccgtccacacgggattaccggcgagtatacatccctcgcaggatcgaagaatacaagttcaatttcattgaagcgaaagcggagcaacttaattgatgatgagaattcatccggatcgttttcatatgacgggatgaagctcggggaagccgagcgttctgcatatggtgactgggccgaggcggagccacccacgtggaaagatttggtattgagggcacgcgtttcctcgatcaatgactctgcttggctttttgattcacaaacatcttcatcatcatttgaatacaacggtgttcccttgggcgagccggaacggcaggctctcagacaatggcaaggagacgctcagcctacctgggaagatctcgttgttaacgcgcgtatggcagaactttgccatgctggttggattgaaggtcaaaaaggttgctttgaagagcgcggggaggctctgcccgcgtcggaacgcggttcgcaacgccccattggtcaacggacagattcctccgattcttttgtgtatgatggcacaaggctcggagcacctgagcgaactgcttatgaacgctggagtaagagggaacgcccgacttgggaagatctcatcttagatgcacaccaggccaggactgaaagtgacgctgttacgacccaagcgattggtcagtcgtcctcaccggttttcttatatgaaggaaagtcgctcggagacagggaacgaaaggcttacgaaaaatggcggcagccagcccaaccgcgatggcaaaatcttgtagtgaacgctcgtctggcagaaatcgatccctcagcctggattgccgatgagcgcgatccgcttgatgatagcgacgcgcttggtcgcccgtcgtacacaagcttgacggatagatcagacgtccctttagacgatcaatcaatctatcgtcgttccgacctagtaagggagcaggtgccagaatcgtctcaaaggcaattcgcagcatgttcagaatctgaaacgaggcctgtgcaatggtttactgcttctgggtcagatgcaaacaatacggaaaatatcaccgccagcgatcccgtcgatcgcacgggtggagttaagcggctaggctccaaaagcgacagaaccgttacagcttctatccatgacgtgaattccagcacaaggcgactgttgcttaacgaatttggatcggaggctccgcgcccttcgccagaaaagactgttcgcttaagaagcgacaatattggcacctatgggagccggaaaaatgaacgagcgcggctcgcgaccgaaaccggtgcgtatgagtcggagcatattttcgggttcaaggctgtccacgatactgcgagagcgacgaaagagggccggcgtctcgaaaggcccatgcccgcctaccttgaggataaggggcttcatcgccaacatattggcaccgggagaggacggaccaaacttgtcgggcgcggatggccggatgacacaagctatcgctcggatcaaagggcaactctgtcggaccccgttgcgcgctcggaaggcgcgacggcctcaaatgggtatcaattgaaccaattgggttacgcgcaccaactcgctagcgatggcctgcaaagtgaatcgcccgatggtgttgccttgccaattcaagtggcaacaacgagctacaactatacagtgagccgcgatcctgtccttgttccgccggataaaaacgaagcccctcaattgctgcatcttggtccccgtggtcaaaccgaagctgttcttgcccgcgaaacagcattgactggaaaatggccgactctcgagcgtgagcagcaagtgtatcgcgagtttttggccttatatgacgtaaaaaaagatcttgaggccaaatcagtcggcgtaagacggaaaaaaaaagaagttatttctgcgttagaccgaactgcgcgcttgataagcacgtcgccttcgaaagctcgatccaaagcagagactgaaaaagccattgatgagctcgatgatcgacgagtttatgatccgcgtgatcgagctcaagacaaagcgtttaaacgctga

SEQ ID NO: 21 - DNA, 198 bp, Agrobacterium  EHA101
atggccatcatcaagccgcatgtgaacaaaaataggacaacctcgccgatagagagaccggagtctctcatagaggaaatgagcggcagtcatccgccgagtggttttaccaacctggatctcgctatgatcgagctggaggactttgtccatcggtgcccgctcccagaagacaatcttgctggtcagaaggagtga

SEQ ID NO: 22 - DNA, 1650 bp, Agrobacterium  EHA101
atggatccgtctagcaatgagaatgtctatgtgggtcgcggtcacaacatcgaaaatgatgatgacactgaccccaggcgttggaagaaggcgaatatcagttccaacaccatctccgatattcagatgacgaatggcgaagacgtacaatcagggagccctacccgaacggaagttgtaagcccacgtctggattatggatcggtcgactcctcctccagcctttattctggcagcgagcacggaaatcaagctgagattcaaaaagagctgtccgtcttgttctcgaacatgtctttgccaggcaacgatcggcgcccggacgaatacattctcgtgcatcaaacgggacaagatgcttttactggtattgccaaaggcaacctcgaccaaatgcccaccaaggcggaatttaacgcgtgctgccgtctctacagggacggagccggtaattactacccgccacctctcgcattcgacaagattagcgttccagagcaactggaggaaaaatgggggatgatggaggcgaaggaacgtaacaaactgcggtttcagtacaagttggacgtatggaatcatgcgcacgctgatatggggatcacgggcacagagatcttttatcaaacagataagaacataaagctcgaccggaattataaactaagacctgaagaccgatacgtacaaacagaaaaatacgggcgccgggaaattcaaaagcgatatcaacacgaactccaggctggttcgctgctgcccgatattatgatcaaaactccccaaaatgacatccacttcgtgtacaggtttgccggcgacaattacgccaacaaacagttcagcgagtttgaacacaccgtcaagcgcaggtatggcgacgagactgagatcaaattgaagtcaaagtcaggcattatgcatgactcgaaatatctggaatcctgggaacggggcagtgcggatattcgcttcgcggaattcgttggggaaaatagagctcacaatcggcagtttccaactgcgacagtaaatatgggacagcagccagacgggcagggcggtttgacccgcgaccgtcatgtgagcgttgacttcctaatgcaaagcgcacccaattcgccttgggcgcaagctttgaaaaagggagaactgtgggatcgcgttcagttgcttgctcgcgacggcaaccgctatctgtcgccgcccagattggaatattctgaccctgcacatttcaccgagttgatgaaccgggttggtttacccgcatcgatgggtcggcaaagccatgcggctagtatcaaattcgaaaagtttgacgcgcaggcagcggttattgtcttaaatggcccagagttacgtgacattcatgacttgtctcctgaaaaactgcaaaatttgtccaccaaagatgtcatcgtcgccgatcgcaatgagaatggtcagagaactggcacgtacaccagcgtcgcggaatatgagcgcttgcagttaaggctgccacccgatgcagcgggggtgcttggtgaagcaactgacaaatattcacgtgatttcgttcggccagagccggcgtcgcgtccaatcagtgacagccgcaggatatacgaaagtcgaccgcgtagccaaagcgtcaacagcttttga

SEQ ID NO: 23 - DNA, 2019 bp, Agrobacterium  EHA101
atggtgaacactacaaagaaaagttttgcgaagtcgcttacggcagatatgcgccgttctgctcagcgcgttgtcgagcaaatgcgaaaagcattgattaccgaagaagaggcgctcaagcggcaagccagactggagagtcccgataggaagcgaaagtatgctgctgatatggcgatagtcgacaaactcgacgtagggtttcgaggcgaaataggctataaaattcttggaaataaccggcttcgagtagacaaccataaagaattaacgcgtgagcacggtagacttcgcaaaaccaaaacggttctgaagcgtaacccggtgacgcaggaagtctatttgggtttatatgaaaggaagtcctggttaagtgtcagcagccatttgtatgctgcggacggcacactccgcatgaagcacgtgaaatacaaagacggacgttttgaggaaaaatgggagcgcgacgaaaatggcgacctgatccgcacaaggtacgccaaccgtggcaggctctttcaacctgtatccgagaaaatgggcgcgccgtatcggagcggccctgacgaccggctctatcgcgatctaacccgtcgaaacggtttcagacgggagacattcgaacgggacgatcacggaaacctcgagcgtatcggcagcaaccatgtcggcttttccaagatttcagtgaaggcacccaatcgtcaaacctcccagacgaagattcaaaaacttggtggcgctttcaacaaatcttttaggtcccttctggacaaggagggcaatgaaatgggccgcgatattttgagccatcgacggctctataacaagcggtctgctgtctacgatgaagctaccggacaattgaagagtgccaagcataccttcggcaagatctacaggagcgaaaccgaatatctcagcgcgggcctcaagaaggtttcaaaaaagatactcggggtgacggtctaccggaaatttgcggcgctcagcgagcgagaatccgaggctgagagactgcgtagttttgaatccggtgcgcatcgccagatctggcaggagcgggcagcgattcccggttcgcccctcccggagactgatgacattcatttcgcacagcagtcgcacctagccaaagccaaccctgatcacgtcgaagctgacgtcacgcgtgtgacagatcaacatgttgatgttgctggacaaacatcatcgtctccccaacggaacttggaaggatggttagattctcaatcacgatacaagccagcaaacatgctgttgtcaaatccagaccttcaagcgaacggacctcgcccatacgaagggttagctcatctcaccctccggcgcgataatgaatctgacgggcacaaggagaatgatcagcggctgcgacatttctcccagccagagccgttggtgttaccgcatcccgggtcgccggaaataactaaggtgtttggctcgcggggagagccggcacacccgagtggaaatctgcacacggcggttggagaaacggcttgcgaaggaccggtgatgtcttcatcctcggacaatcatcagccagctccaggacagcaagaacttttaagtttccttcataatgcgccagccccagtttctgtggcaatacatgatgatcaagagcgacttgcgggggaggcgcccggcggctctttcagaggtagctcagggcgaacgagttcaatgtcggagagtatcttcgacgaagatgtacaagggcatttggtacgggattattcgatcaatactactaacgggtttattgacccgcaatcgttgttcggtgaaccggacttatcgagaggtccaaaatcggggccagaaattccatcggaagattaccatttgtcagcttcggaacaggaaaatttgctgaatcaattgcttagtgtgccactgccggttccttcaccgaagcccgaatgcgcgaggtctatgattttcgaaggttcacgttcaagagagcgttccacctccagagggttctaa

SEQ ID NO: 24 - DNA, 438 bp, Agrobacterium  EHA101
atgaaacgtatcagtacaatccttgtcggcgtatttttggctgcgccggtttatgcagccgacaatattcatacattagggacgttgtctgaaattgaactagcgctcactgccggaaagccggtgaacgtcacggttgatttaagtttgtgtgctcccggggtggccgatactccggcgacgaaaacgcaaggaggcatgcgcatagatgcgtatcgaataacaactgacggcacccttgcgtttgcagatcagcacttcaccattgaccgtgacggtaaacccataactcaatttattcgttatcagattcggtcaaacggtgacgccgatttcacgatggttacattcaacatgccaacatatgagcgaaaaggtaccagcttggcctacaagtgcgcgatcgaccacgggttgagttttcgtactccacagtga

SEQ ID NO: 25 - DNA, 696 bp, Agrobacterium  EHA101
tcaagcctccaagattttgctgatcagtttcgaaatgaccacgacttcctccatcgcaatccgaagattcctctctatgaggcgcatcgtcggatcagttcccgtgtttagtaatgtaagatgcaacatgccgcgttctttcatcgcggcaaatgcatctctttcatgcatgggagacggtacaactggaaggctctctagcgtctctgacatcctgcgttgcgatgttgtcaatcggccgaccgggacgcgttggcgcaaaacagctgtaggaattgccaaattttcactcaacagcagctcgatgacgtagcggtaggtagatagtgcctcatcgatgtcgagcggcgttagcatggtggggatcagaagcaggtttgagctagcgatgattgtgttgttgagctcgctcgagccgccacgcgtatcggccaacgcataatcaaatccttcgagctcggcattttcataggctgcttcaagaaggggcatttcgtcggcggaatagacttcacagcgaggatcccaggtactgctttgtaaggcgttttctctccatcgcgtcagaggccggttttcgtcggcatcaaagagggccactcgtttaccgtcatttgccaaagcagcgcaaaggcccatgagtgcggtggttttgccagcaccccctttgaaagaacaaaacgtcaaaagttgcat

SEQ ID NO: 26 - DNA, 20 bp, Artificial Sequence
  Description of Artificial Sequence Synthetic DNA
ttagcgcctctgaaattcgg

SEQ ID NO: 27 - DNA, 21 bp, Artificial Sequence
  Description of Artificial Sequence Synthetic DNA
aaggcacttcgtcagcgacca

SEQ ID NO: 28 - DNA, 22 bp, Artificial Sequence
  Description of Artificial Sequence Synthetic DNA
ttacttctgaccgcaagagatc

SEQ ID NO: 29 - DNA, 20 bp, Artificial Sequence
  Description of Artificial Sequence Synthetic DNA
ttcgccagatgctcagcgat

SEQ ID NO: 30 - DNA, 21 bp, Artificial Sequence
  Description of Artificial Sequence Synthetic DNA
ggtggttggaaagctcatgtc

SEQ ID NO: 31 - DNA, 20 bp, Artificial Sequence
  Description of Artificial Sequence Synthetic DNA
cgatccagaaaatcctggcg

SEQ ID NO: 32 - DNA, 20 bp, Artificial Sequence
  Description of Artificial Sequence Synthetic DNA
caccggcttctgcgacgcgc

SEQ ID NO: 33 - DNA, 22 bp, Artificial Sequence
  Description of Artificial Sequence Synthetic DNA
aatctatccgtagatcatcctc

SEQ ID NO: 34 - DNA, 19 bp, Artificial Sequence
  Description of Artificial Sequence Synthetic DNA
gtggatatcgaggggacac

SEQ ID NO: 35 - DNA, 20 bp, Artificial Sequence
  Description of Artificial Sequence Synthetic DNA
acatactttcatcgaagtcg

SEQ ID NO: 36 - DNA, 20 bp, Artificial Sequence
  Description of Artificial Sequence Synthetic DNA
ggtcaccgttccgttcggtt

SEQ ID NO: 37 - DNA, 21 bp, Artificial Sequence
  Description of Artificial Sequence Synthetic DNA
tctccgcgaacagatctactg

SEQ ID NO: 38 - DNA, 20 bp, Artificial Sequence
  Description of Artificial Sequence Synthetic DNA
caattgactgacctcgcgaa

SEQ ID NO: 39 - DNA, 20 bp, Artificial Sequence
  Description of Artificial Sequence Synthetic DNA
cgtgcatgctccttctttct

SEQ ID NO: 40 - DNA, 20 bp, Artificial Sequence
  Description of Artificial Sequence Synthetic DNA
ttcctggatccaccgcgctg

SEQ ID NO: 41 - DNA, 20 bp, Artificial Sequence
  Description of Artificial Sequence Synthetic DNA
atcggctcaattgatatccc

SEQ ID NO: 42 - DNA, 20 bp, Artificial Sequence
  Description of Artificial Sequence Synthetic DNA
tttgcttggtcacctgaaac

SEQ ID NO: 43 - DNA, 20 bp, Artificial Sequence
  Description of Artificial Sequence Synthetic DNA
ccgcacttacgtcctcgtac

SEQ ID NO: 44 - DNA, 22 bp, Artificial Sequence
  Description of Artificial Sequence Synthetic DNA
agttgaggatgtttttcaggag

SEQ ID NO: 45 - DNA, 21 bp, Artificial Sequence
  Description of Artificial Sequence Synthetic DNA
tagggtcggttactctaggat

SEQ ID NO: 46 - DNA, 20 bp, Artificial Sequence
  Description of Artificial Sequence Synthetic DNA
aaaccgcactcccgatattc

SEQ ID NO: 47 - DNA, 20 bp, Artificial Sequence
  Description of Artificial Sequence Synthetic DNA
cactcggtccttctctatct

SEQ ID NO: 48 - DNA, 20 bp, Artificial Sequence
  Description of Artificial Sequence Synthetic DNA
aggctgtgttgttcgcagca

SEQ ID NO: 49 - DNA, 22 bp, Artificial Sequence
  Description of Artificial Sequence Synthetic DNA
ccaaagattggccccttgatac

SEQ ID NO: 50 - DNA, 20 bp, Artificial Sequence
  Description of Artificial Sequence Synthetic DNA
gttggttcccaatgccaatc

SEQ ID NO: 51 - DNA, 20 bp, Artificial Sequence
  Description of Artificial Sequence Synthetic DNA
cacttcaccgagattcttcg

SEQ ID NO: 52 - DNA, 20 bp, Artificial Sequence
  Description of Artificial Sequence Synthetic DNA
cttcaagctgcctttcacat

SEQ ID NO: 53 - DNA, 20 bp, Artificial Sequence
  Description of Artificial Sequence Synthetic DNA
aagatcaaaggcgactcctc

SEQ ID NO: 54 - DNA, 20 bp, Artificial Sequence
  Description of Artificial Sequence Synthetic DNA
gcctttcacattggaatcat

SEQ ID NO: 55 - DNA, 20 bp, Artificial Sequence
  Description of Artificial Sequence Synthetic DNA
atgggaagacctcgttgtca

SEQ ID NO: 56 - DNA, 20 bp, Artificial Sequence
  Description of Artificial Sequence Synthetic DNA
aaggtcgtccacgatacttt

SEQ ID NO: 57 - DNA, 20 bp, Artificial Sequence
  Description of Artificial Sequence Synthetic DNA
ggcactgctgtcaagaaatc

SEQ ID NO: 58 - DNA, 20 bp, Artificial Sequence
  Description of Artificial Sequence Synthetic DNA
attcctgagcattgaggtcc

SEQ ID NO: 59 - DNA, 20 bp, Artificial Sequence
  Description of Artificial Sequence Synthetic DNA
tgctttctaggctgctgcag

SEQ ID NO: 60 - DNA, 20 bp, Artificial Sequence
  Description of Artificial Sequence Synthetic DNA
atggagcaaaccttaatttg

SEQ ID NO: 61 - DNA, 20 bp, Artificial Sequence
  Description of Artificial Sequence Synthetic DNA
ggacatcccggtggaattta

SEQ ID NO: 62 - DNA, 20 bp, Artificial Sequence
  Description of Artificial Sequence Synthetic DNA
accgaaggctcaattctatt

SEQ ID NO: 63 - DNA, 20 bp, Artificial Sequence
  Description of Artificial Sequence Synthetic DNA
atcattgtttctcctacaga

SEQ ID NO: 64 - DNA, 20 bp, Artificial Sequence
  Description of Artificial Sequence Synthetic DNA
aaggagttagacgatggatc

SEQ ID NO: 65 - DNA, 19 bp, Artificial Sequence
  Description of Artificial Sequence Synthetic DNA
cagctcgcattgaattccg

SEQ ID NO: 66 - DNA, 19 bp, Artificial Sequence
  Description of Artificial Sequence Synthetic DNA
acctggcggcaaaccgaca

SEQ ID NO: 67 - DNA, 20 bp, Artificial Sequence
  Description of Artificial Sequence Synthetic DNA
tagccggctaggttttcttc

SEQ ID NO: 68 - DNA, 19 bp, Artificial Sequence
  Description of Artificial Sequence Synthetic DNA
ggtcgacacgacgaagaag

SEQ ID NO: 69 - DNA, 20 bp, Artificial Sequence
  Description of Artificial Sequence Synthetic DNA
tatttcacatgcttcgcgcg

SEQ ID NO: 70 - DNA, 20 bp, Artificial Sequence
  Description of Artificial Sequence Synthetic DNA
accgcgtcagtgacgaagcc

SEQ ID NO: 71 - DNA, 20 bp, Artificial Sequence
  Description of Artificial Sequence Synthetic DNA
gcgttacgcttcagaacctt

SEQ ID NO: 72 - DNA, 19 bp, Artificial Sequence
  Description of Artificial Sequence Synthetic DNA
tgaaacaggccaagatgtg

SEQ ID NO: 73 - DNA, 20 bp, Artificial Sequence
  Description of Artificial Sequence Synthetic DNA
tcattcaccatcgcgcgcca

SEQ ID NO: 74 - DNA, 20 bp, Artificial Sequence
  Description of Artificial Sequence Synthetic DNA
atcaattgagccgatcttcc

SEQ ID NO: 75 - DNA, 20 bp, Artificial Sequence
  Description of Artificial Sequence Synthetic DNA
aggtgatcctatttagattg

SEQ ID NO: 76 - DNA, 19 bp, Artificial Sequence
  Description of Artificial Sequence Synthetic DNA
tgatctctgactcctgtgt

SEQ ID NO: 77 - DNA, 20 bp, Artificial Sequence
  Description of Artificial Sequence Synthetic DNA
cttccatgtgctgatttcca

SEQ ID NO: 78 - DNA, 20 bp, Artificial Sequence
  Description of Artificial Sequence Synthetic DNA
tctctgcgtcgatttcaaga

SEQ ID NO: 79 - DNA, 20 bp, Artificial Sequence
  Description of Artificial Sequence Synthetic DNA
tccttatcctgtcgattttg

SEQ ID NO: 80 - DNA, 20 bp, Artificial Sequence
  Description of Artificial Sequence Synthetic DNA
cagatgggaattcgcaaacc

SEQ ID NO: 81 - DNA, 20 bp, Artificial Sequence
  Description of Artificial Sequence Synthetic DNA
tgtgtgagggatcggataac

SEQ ID NO: 82 - DNA, 20 bp, Artificial Sequence
  Description of Artificial Sequence Synthetic DNA
ctacgcgcatgaagatcaag

SEQ ID NO: 83 - DNA, 20 bp, Artificial Sequence
  Description of Artificial Sequence Synthetic DNA
tcctcgcccttgctcaccat